(19) United States
(12) Patent Application Publication
(10) Pub. No.: US 2017/0354609 A1
(43) Pub. Date: Dec. 14, 2017

(54) PHARMACEUTICAL TABLET COATING PROCESS BY INJECTION MOLDING PROCESS TECHNOLOGY

(71) Applicant: Massachusetts Institute of Technology, Cambridge, MA (US)

(72) Inventors: Vibha Puri, San Francisco, CA (US); Parind Mahendrakumar Desai, Quincy, MA (US); Keith D. Jensen, Arlington, MA (US); David Brancazio, Cambridge, MA (US); Eranda Harinath, Waltham, MA (US); Alexander Racine Martinez, Somerville, MA (US); Jung Hoon Chun, Sudbury, MA (US); Richard Dean Braatz, Arlington, MA (US); Allan S. Myerson, Boston, MA (US); Bernhardt Levy Trout, Lexington, MA (US)

(73) Assignee: Massachusetts Institute of Technology, Cambridge, MA (US)

(21) Appl. No.: 15/620,568

(22) Filed: Jun. 12, 2017

Related U.S. Application Data
(60) Provisional application No. 62/348,371, filed on Jun. 10, 2016.

(51) Int. Cl. A61K 9/28; A61K 9/20; A61K 31/343; B29C 45/14; B29K 67/00; B29K 71/00; B29K 29/00

(52) U.S. Cl. CPC A61K 9/2893; A61K 31/343; A61K 9/2853; A61K 9/205; B29C 45/14819; A61K 9/2018; A61K 9/284; A61K 9/2013; B29K 2071/02; B29K 2029/04; B29K 2067/04

(57) ABSTRACT

The disclosure describes an injection molding process for coating a tablet core to produce a coated pharmaceutical tablet, wherein the injection-molded coating is substantially continuous (e.g., completely covers the tablet core with no openings), and describes the resulting coated pharmaceutical tablet. The disclosure describes compositions for coatings and tablet cores and equipment suitable for performing the process.

### RELATED APPLICATIONS

This application claims priority to U.S. Provisional Application No. 62/348,371, filed Jun. 10, 2016, which is incorporated herein by reference in its entirety.

### FIELD OF INVENTION

The present invention generally relates to pharmaceutical tablets and methods of making same.

### BACKGROUND OF INVENTION

Tablet coating is a common unit operation in tablet manufacturing in the pharmaceutical industry. Currently, the operation is generally done as a batch process. Tablets are generally coated by spraying or ladling the coating solutions on the tablet surface. Tablet coating is conventionally done using the spray coating process in the pharmaceutical industry. This is typically performed in a batch process, e.g. in a drum coater wherein aqueous or non-aqueous solvent-based coating liquid (solution/suspension) is spray-coated onto tablets in a rotating perforated coating drum, with simultaneous drying by heated air. Furthermore, this coating methodology is typically employs different aqueous and/or organic solvents to facilitate coat spraying or ladling.

Tablet coating is one of the most common pharmaceutical unit operations, providing benefits such as taste masking, odor masking, physical and chemical protection, product differentiation, and elegant appearance Achieving tailored drug release profiles and separation of incompatible drugs into separate coat and core formulations are other advantages of tablet coating. Tablet coating reduces dust generation and friction that can further decrease tablet friability and increase packaging speed.

Film coating involving organic and aqueous solvent based polymer systems is the most commonly used tablet coating technology. The organic solvents used can be expensive, flammable and toxic in nature. Strict environmental regulations, possible safety hazards to the instrument operator, costly solvent recovery system and possibility of residual solvent in final formulation further complicate the acceptability of organic solvents in coating.

### SUMMARY OF INVENTION

Coated pharmaceutical tablets and injection molding processes for making or coating the same are generally provided.

According to some embodiments, methods are provided for manufacturing and/or coating a tablet.

According to one or more embodiments, a method for coating a pharmaceutical tablet comprises: positioning a tablet core in an injection mold cavity; and injecting a coating composition into the injection mold cavity to form an injection-molded coating on the tablet core and to produce a coated pharmaceutical tablet, wherein the injection-molded coating is substantially continuous.

According to one or more embodiments, a method for coating a pharmaceutical tablet comprises: positioning a tablet core in a first orientation in an injection mold cavity; injecting a coating composition into the injection mold cavity to form an injection-molded coating on a first portion of the tablet core; reorienting the tablet core with respect to the injection mold cavity; and injecting the coating composition into the injection mold cavity to form an injection-molded coating on a second portion of the tablet core to produce a coated pharmaceutical tablet, wherein the injection-molded coating is substantially continuous.

According to one or more embodiments, a method for manufacturing a coated pharmaceutical tablet comprises: injecting a coating composition into an injection mold cavity to form a first portion of an injection-molded coating; injecting a tablet core composition into the injection mold cavity onto the first portion of the injection-molded coating to form a partially-coated tablet core; and injecting the coating composition into the injection mold cavity onto the partially-coated tablet core to produce a coated pharmaceutical tablet, wherein the injection-molded coating is substantially continuous.

According to some embodiments, coated pharmaceutical tablets are provided.

According to one or more embodiments, a coated pharmaceutical tablet comprises: a tablet core comprising one or more layers; and an injection-molded coating surrounding the tablet core, wherein the injection-molded coating is solvent-free and substantially continuous, and wherein the injection-molded coating comprises a coating composition.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A shows a representation of an injection molding unit according to one set of embodiments of the invention;

FIG. 1B shows a representation of an injection molding unit according to one set of embodiments of the invention;

FIG. 1C shows a representation of a coated pharmaceutical tablet according to one set of embodiments of the invention;

FIG. 2 shows a representation of a coated pharmaceutical tablet according to one set of embodiments of the invention;

FIG. 3 show images of a coated pharmaceutical tablet according to one set of embodiments of the invention;

FIG. 4A shows an image of a pharmaceutical tablet according to one set of embodiments of the invention;

FIG. 4B shows an image of a pharmaceutical tablet according to one set of embodiments of the invention;

FIG. 4C shows an image of a pharmaceutical tablet according to one set of embodiments of the invention;

FIG. 5 shows a chart depicting dissolution profiles of pharmaceutical tablets according to one set of embodiments of the invention;

FIG. 6A shows an image of a pharmaceutical tablet according to one set of embodiments of the invention;

FIG. 6B shows an image of a pharmaceutical tablet according to one set of embodiments of the invention;

FIG. 6C shows an image of a pharmaceutical tablet according to one set of embodiments of the invention;

FIG. 6D shows an image of a pharmaceutical tablet according to one set of embodiments of the invention;

FIG. 7A shows an image of a pharmaceutical tablet according to one set of embodiments of the invention;

FIG. 7B shows an image of a pharmaceutical tablet according to one set of embodiments of the invention; and

FIG. 8 shows a chart depicting dissolution profiles of pharmaceutical tablets according to one set of embodiments of the invention.

### DETAILED DESCRIPTION

According to one or more embodiments, processes and compositions are disclosed for coating tablet cores to produce coated pharmaceutical tablets. The coating of pharmaceutical tablets may provide additional functionality to the drug product. This includes functionality such as product identification, improved stability (providing protection from environmental stressors such as moisture and light), improved mechanical integrity, and the ability to modify the drug release rate from tablet cores, to impart taste masking, etcetera.

According to one or more embodiments coated pharmaceutical tablets are disclosed. The tablet may comprise a tablet core, and an injection-molded coating surrounding the tablet core, wherein the injection-molded coating is solvent-free and substantially continuous, and wherein the injection-molded coating comprises a coating composition comprising at least one polymer and at least one plasticizer.

According to one or more embodiments, a tablet coating process may use an injection molding (IM) process technology. The injection molding process may involve use of specific coat molds (e.g., coat mold inserts) to define the tablet coat dimensions and provided desired coat characteristics such as shape and thickness. According to some embodiments, during the process, the molten coating composition may be injected into an injection mold cavity containing the tablet core. The coating composition may solidify and form a film or coating on the tablet core. The injection-molded coating may be substantially continuous, in other words, fully surrounding the tablet core with no holes or openings in the coating exposing the core.

According to some embodiments, a pharmaceutical tablet coating process may comprise at least two injecting steps. The tablet core may be positioned in a first orientation in an injection mold cavity. A coating composition may then be injected into the injection mold cavity to form an injection-molded coating on a first portion of the tablet core. The tablet core (now partially coated) may be reoriented with respect to the injection mold cavity. The coating composition may then be injected into the injection mold cavity to form an injection-molded coating on a second portion of the tablet core to produce a coated pharmaceutical tablet, wherein the injection-molded coating is substantially continuous. In some embodiments, the tablet core may be reoriented with respect to the injection mold cavity by physically repositioning (e.g., inverting) the tablet core within the injection mold unit, either manually or through an automated process. In some embodiments, reorientation may involve a reconfiguration of the injection molding device around the tablet. Details of such two-step coating methods are described in further detail herein.

According to some embodiments, a previously formed tablet core may be introduced to an injection molding unit prior to any coating being applied to it. According to other embodiments, the tablet core may be formed by injection molding it onto a portion of the coating already within the injection molding unit. For example, according to some embodiments, a coated pharmaceutical tablet may be produced by first injection molding one portion of the coating onto the tablet, chilling this portion, or, allowing it to solidify, and then injection molding the core inside the half coat, followed by injection molding of the other half of the coat.

Use of injection molding technology for tablet coating can provide many process and product advantages. Tablet coating by injection molding may be a solvent-free process, in some embodiments. This is advantageous for solvent-sensitive drug products and eliminates the need to remove toxic residual solvents.

During the injection molding coating process, the tablet core is exposed to the molten coating material for only a very short time period (in the range of 1 to 10 seconds), in some embodiments. In comparison, there is prolonged exposure of tablets to heated air during a spray coating process. This prolonged exposure may have detrimental effects.

The injection molding process has the potential to be developed into a continuous or semi-continuous manufacturing process, in some embodiments, which allows for more efficient manufacturing.

The injection molding coating can provide for improved process control. For example, by designing coat molds of variable thickness and designs, the performance of the coating, and thus drug release, may be precisely modulated.

The injection molding process obviates the need of a drying step in the coating process, in some embodiments.

The coating can easily be designed to be as thin or thick as desired providing increased flexibility in the function of the coat compared to a traditional spray coating, according to some embodiments.

According to certain embodiments, the tablet coating processes described herein may be set up as a continuous manufacturing process. Transformation from batch to continuous manufacturing can yield a commercial advantage in terms of total operation cost and improved product quality by real-time control of process. In batch manufacturing, the final product is traditionally manufactured with several individual and separated sequence of batch-wise unit operations. This can result in inefficient and delayed processing with more chances of processing errors, defects in final product, and typically require a 14-24 months manufacturing cycle time. Continuous manufacturing is an uninterrupted processing technology that can be implemented to be a seamless flow of production. It reduces processing time and could provide more reliable products with smaller equipment footprint, less scale-up requirement and reduced production costs.

Injection molding (“IM”) is a rapid, melt processing-based and versatile technology to manufacture products of diverse and intricate three dimensional shapes with high precision. The quality of an IM product relies on different factors such as part design, mold design, material attributes and process parameters. Process parameters such as injection pressure, hold pressure, mold surface temperature, and cooling time aid in achieving a robust IM product.

According to certain embodiments, an injection molding unit may be used to perform the above and alternative processes related to coating and manufacturing tablets. In some embodiments, the unit may be either vertical- or horizontal-opening injection molding unit, or orientated on any other axis or mode of operation. According to certain embodiments, the unit may comprise an injection piston, temperature controlled injection barrel, temperature controlled mold cavity comprising two mold halves and orifice, and an adjustable ejection pin. The unit may have a process control and data acquisition system to control and monitor process parameters. In some embodiments, interchangeable mold-base inserts may be used for the different coating stages. Circulating fluid may be used to maintain a desired temperature. An adjustable ejector pin was installed for working with mold inserts of different depths.

According to certain embodiments, specific mold inserts may be created to form a desired coating for a given tablet core. To develop the coat mold tooling, the first step is to accurately characterize the dimensions of the ‘core’ extrusion-molded tablets. Based on this, coating mold inserts for the target coat thickness were designed to complement the ‘core’ tablet shape.

Heat melt extrusion (“HME”) is a continuous melt processing technology that is widely used in the plastic industry and involves the mixing of polymers, carriers and other constituents with the application of heat and shear.

In some embodiment, HME may be used to prepare compositions for delivery to the injection molded unit. In some embodiments, an integrated HME-IM system may comprise a set-up of a hot melt twin screw extruder coupled to a horizontally opening injection mold machine to generate cores and/or coatings of tablets.

A number of process parameters affect the heat extrusion and injection molding process. A process control system may be used to guide these parameters. According to certain embodiments, the coating process and coat compositions can be controlled to include a range of coat mold designs, provide different functionality coats (e.g. moisture protection, taste masking) and achieve targeted drug release profiles. For example, the IM process parameters of injection pressure, injection time, hold pressure, and hold time may be controlled with software (e.g., a LabVIEW program). In some embodiments, a control algorithm is used to track and control a given parameter for the HME and/or IM process.

Extrusion parameters that may affect the desired formation of an injection mold feed stock include, without limitation: feed flow rate; screw speed; feed zone temperature; extruder barrel temperature, etc. The desired values for these parameters may depend on the components of feed and the desired properties of the final product. Values other than those listed below are possible.

In some embodiments, the extrusion feed flow rate may be from about 1 g/hr to about 500 g/hr. In some embodiments the feed flow rate is about 80 g/hr. In some embodiments, the extrusion screw speed may be from about 10 RPM to about 1200 RPM, more preferably from about 50 RPM to about 150 RPM. In some embodiments the screw speed is about 90 RPM.

In some embodiments, the feed zone temperature may be from about −20° C. to about 300° C., more preferably from about 0° C. to 100° C., or even more preferably about 5° C. to 50° C. In some embodiments the feed zone temperature is about 8° C.

In some embodiments, the extruder barrel temperature may be about from about 25° C. to about 300° C., more preferably from about from about 100° C. to about 160° C. The extrusion temperature may be optimized to provide proper flow of the melted coating composition without degradation of the materials such as excipients or a drug in the coating materials or in the core tablet; and without providing a composition so hot that the tablet becomes melted or damaged when the extruded coating composition is brought into contact with it.

According to certain embodiments, injection molding parameters that may be controlled or monitored, via a process control and data acquisition system, include, without limitation: injection pressure, injection speed, barrel temperature, mold temperature, and hold time, among others.

The injection pressure employed to inject a composition (e.g., coating composition) from the barrel to the mold cavity is an important parameter. Low injection pressure could result in incomplete mold filling, whereas, high injection pressure could generate pressure induced stress and flashing. In preferred embodiments, injection pressure should be controlled such that the pressure does not oscillate during the injection cycle. Oscillation may cause the deformation of tablet cores whereas, unfluctuating pressure pattern eliminated core deformation when optimized pressure and temperature profiles were employed. According to certain embodiments, injection pressure may range from 100 PSI to 40,000 PSI, preferably 1000 PSI to 30,000 PSI. Hold pressure may range from 0 PSI to 44,000 PSI, preferably 1100 PSI to 33,000 PSI.

The barrel temperature of the IM device is another important parameter to ensure proper flow of the material and therefore the IM product quality. The barrel temperature may be optimized to achieve a complete tablet coat at the lowest temperature and injection pressure. Barrel temperature may range from 40° C. to 300° C., preferably 60° C. to 150° C.

Mold temperature and cooling time are parameters that may be optimized with the aim to minimize the differences between the barrel and mold temperatures without the cooling time being excessively long. The quality of the final product is affected by the cooling stage of the injection molding cycle wherein a hot melted polymer is injected into the mold and allowed to stay until it solidifies. Optimization of cooling time and the mold temperature plays an important role in providing a good coating. During the cooling stage, heat transfer also affects crystallization kinetics, shrinkage, and residual stresses and thereby impacts the mechanical properties, surface clarity and geometric tolerance. Hold time (i.e., cooling time or solidification time) may range from 0 s to 120 s, preferably 2 s to 30 s. Mold temperature may range from 0° C. to 150° C., preferably 20° C. to 100° C.

In some embodiments, a higher injection flow rate promotes a better weld between the two portions of the coating. Injection flow rate may range from 0.1 ml/sec to about 20 ml/sec. Other values are also possible. Injection time may range from 0.1 s to 60 s, preferably 0.5 s to 20 s.

Humidity during processing may also affect performance of the final coating. In some embodiments, IM coating compositions require sufficient room humidity (e.g., greater than 30% RH) to avoid immediate cracks, whereas other formulations are insensitive to the room humidity.

The above described parameters may be incorporated into one step or multi-step processes for forming a tablet core and/or tablet coating. According to one or more embodiments, a one-step injection molding process may be implemented. The process may comprise positioning a tablet core in an injection mold cavity. A coating composition is then injected into the injection mold cavity to form an injection-molded coating on the tablet core and to produce a coated pharmaceutical tablet.

According to some embodiments, a pharmaceutical tablet coating process may comprise at least two injecting steps. The tablet core may be positioned in a first orientation in an injection mold cavity. A coating composition may then be injected into the injection mold cavity to form an injection-molded coating on a first portion of the tablet core. The tablet core (now partially coated) may be reoriented with respect to the injection mold cavity. The coating composition may then be injected into the injection mold cavity to form an injection-molded coating on a second portion of the tablet core to produce a coated pharmaceutical tablet, wherein the injection-molded coating is substantially continuous. As noted above, the tablet core may be reoriented with respect to the injection mold cavity by repositioning the tablet core within the injection mold unit or by a reconfiguration of the injection molding device.

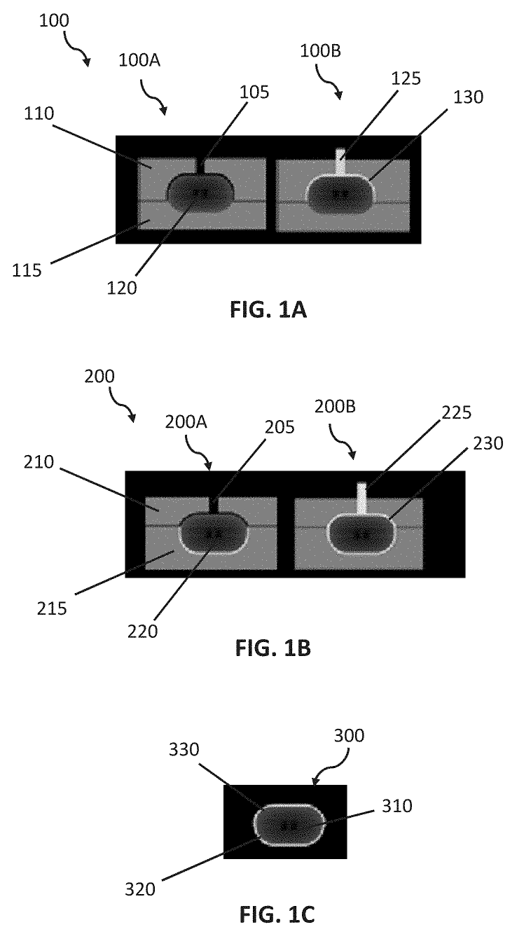

FIGS. 1A-1C illustrate a schematic of the injection molding unit during a representative two-step process and the produced coated pharmaceutical tablet. FIG. 1C shows a finished coated pharmaceutical tablet 300 resulting from the two-step process illustrated in FIGS. 1A and 1B. In one embodiment, the tablet core 310 may have a non-symmetrical shape that for the sake of simplicity and clarity may be referred to as a “pot and lid” shape. In other embodiments, it may be symmetrical. Formed by the step illustrated by FIG. 1A, a first coating portion 320 coats the pot portion of the core 310. Formed by the step illustrated by FIG. 1B, a second coating portion 330 coats the lid portion of the core 310. The two coating portions 320 and 330 form a continuous whole with no openings exposing the tablet core 310.

FIG. 1A shows an injection mold unit in a first arrangement 100 prior to (100A) and during (100B) a first step of injecting coating composition. The unit 100A comprises an upper mold insert 110 and a lower mold insert 115. The use of the terms “upper” and “lower” is purely for the sake of convenience and clarity—alternative configurations could just as well be applied. The mold inserts define a cavity into which a tablet core 120 may be placed in a first orientation (e.g., having a first side facing up). An orifice 105 connects the cavity to a source of coating composition 125 that is used to form a coat 130.

FIG. 1B shows the injection molding unit in a second arrangement 200 prior to (200A) and during (200B) a second step of injecting coating composition. In FIG. 1B, the partially coated tablet core 220 is repositioned in a second orientation in the injection mold cavity. Here the tablet 220 has been inverted or flipped over. A new set of upper and lower mold inserts 210 and 215 are placed into the unit to provide an altered geometry of the cavity. (In some embodiments insert 215 may be the same as mold insert 110, with a pin in the channel 105 for non-overlapping coat designs.) The new inserts 210 and 215 account for the fact that the tablet shape has been slightly altered to the partial coating it received during the first step. Furthermore, the tablet need not be symmetrical and therefore the portion of the tablet 220 receiving the second coating 230 may have a different shape requiring different mold inserts 210 and 215. Coating composition material 225 (generally, the same as composition 125, but potentially different) is fed through orifice 205 to provide the completed coating 230. The completed coating is substantially continuous, meaning that the tablet 300 has no openings in its coating.

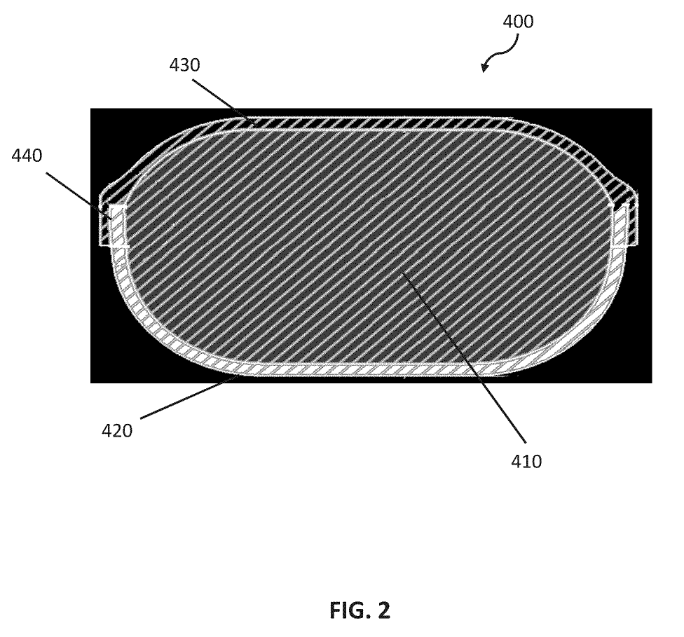

FIG. 2 shows an example of a coated pharmaceutical tablet 400 having an overlapping region 440 in the coating. The tablet 400 includes a core 410 that generally comprises active pharmaceutical ingredient(s) and excipient. The core 410 has a “pot and lid” shape with the lower half (as shown) constituting the pot and the upper half the lid. Of course, other geometries may also be employed. The coating of the tablet 400 was formed through a two-step process like that described with regard to FIGS. 1A-1C. A first coating 420 was applied to the pot portion of the core 410 and then a second coating 430 was applied to the lid portion of the core 410. The mold inserts were configured to cause the second coating 430 to overlap the first coating 420 in an overlapping region 440. In some embodiments, use of an overlapping region 440 helps strengthen the weld between the two coating portions 420 and 430. However, in other embodiments, the overlap may be avoided while still providing a robust coating.

According to one or more embodiments, tablet cores used may be formed from a variety of methods. For example, tablet cores may be machined or injection molded, made through powder/granule compaction or compression, or through other techniques whether now known or later developed.

According to some embodiments, a process for injection molding the entire tablet (e.g., both core and coating) may be implemented. The process may comprise injecting a coating composition into an injection mold cavity to form a first portion (e.g., first half) of an injection-molded coating. A tablet core composition may then be injected into the mold cavity onto the first portion of the injection-molded coating to form a partially-coated tablet core. Additional coating composition may then be injected into the injection mold cavity onto the partially-coated tablet core to produce a coated pharmaceutical tablet, wherein the injection-molded coating is substantially continuous.

According to one or more embodiments, the tablet core may comprise one or more layers. In some embodiments, the tablet core may comprise a single layer having a uniform composition. In some embodiments, the tablet core may comprise multiple layers, with different layers having different compositions. The tablet core may comprise one or more excipient materials. The tablet core may comprise excipient material now known or later developed. Examples of common excipients include, without limitation, the following: maltodextrin, xylitol, lactose, mono- and di-calcium phosphate, mannitol, sorbitol, magnesium stearate, hypromelose, microcrystalline cellulose and other cellulose derivatives, starch and modified starches, calcium carbonate, polyethylene oxides, acrylates, polyvinyl alcohols, polyvinyl alcohol/polyethylene glycol graft copolymers, polyethylene glycols, polyvinyl acetates, polyvinylcaprolactam-based graft copolymers.

According to one or more embodiments, the tablet core may further comprise a pharmaceutically active agent, now known or later developed. In the examples below, griseofulvin (GF) serves as an active ingredient.

According to certain embodiments, the coating composition may comprise a combination of coating polymers and plasticizers in different concentrations. According to certain embodiments, the coating composition comprises 50% to 100% by weight polymer, and 0% to 50% by weight plasticizer. According to certain embodiments, the coating composition may comprise 60% to 100% by weight polymer, and 0% to 40% by weight plasticizer. In some embodiments, 10%, 20%, 30%, or 40% by weight polymer may be employed. Other materials in the coating composition may include disintegrants, fillers, lubricants, etc.

Preferred coating compositions may also be characterized by certain mechanical properties. For example, in some embodiments it has been found that a suitable composition has one or more of the following properties: less than 700 MPa Young's modulus, greater than 30% elongation, greater than 95×10⁴ J/m³ toughness. and melt flow of greater than 0.4 g/min. Testing methods to determine values for these and other mechanical characteristics of the coating compositions and tablet cores are discussed in the Examples below.

According to certain embodiments, coating polymers used may include polyethylene oxide, acrylates, polyvinyl alcohol, polyvinyl alcohol/polyethylene glycol graft copolymer, polyethylene glycol, polyvinyl acetate and polyvinylcaprolactame-based graft copolymer, hydroxypropyl peastarch, and polyetherimide.

According to certain embodiments, plasticizers used may include glycerine, glyceryl behenate, polyethylene glycols, acetyltributyl citrate, acetyltriethyl citrate, benzyl benzoate, castor oil, chlorobutanol, diacetylated monoglycerides, dibutyl sebacate, diethyl phthalate, mannitol, polyethylene glycol monomethyl ether, propylene glycol, polysorbates (tweens), Pullulan, sorbitol, sorbitol sorbitan solution, triacetin, tributyl citrate, glacial triethyl citrate, vitamin E, water and others.

According to certain embodiments, the polymer comprises polyethylene oxide and the plasticizer comprises polyethylene glycol. According to certain embodiments, the polymer comprises an acrylate-based polymer and the plasticizer comprises an acrylate-based plasticizer. According to certain embodiments, the polymer comprises polyvinyl alcohol and the plasticizer comprises glycerine or polyethylene glycol plasticizer. According to certain embodiments, the polymer comprises a graft copolymer of polyvinyl alcohol and polyethylene glycol and the plasticizer comprises glycerine or polyethylene glycol. According to certain embodiments, the polymer comprises polyethylene glycol and a graft copolymer of polyvinyl acetate and polyvinylcaprolactame-based polymer and the plasticizer comprises glycerine or polyethylene glycol.

Plasticizer may be added to the coating polymers to improve flow and processability and reduce the brittleness of the coating polymer. Plasticizer interposes its molecules between the polymer chains and can also bond with the functional groups of the polymer chains. Thus, it reduces the interaction between the polymer chains and increases the volume between them, imparting chain mobility and flexibility or distensibility.

Plasticizer may be selected and optimized to satisfy certain parameters. For example, plasticizer may be selected and optimized to provide a desired dissolution time of the resulting coated tablet. Dissolution time may be understood to be the time necessary for 75% drug release. Plasticizer may be selected and optimized to provide a melt viscosity of the polymer/plasticizer formulation during processing, allowing in turn for the process parameters of injection temperature and injection pressure to have preferred values. Plasticizer may be selected and optimized to yield homogenous polymer-plasticizer matrix during processing to achieve acceptable coat film properties.

Added plasticizer may be selected to be compatible and preferably miscible with the polymer, and so most of the selected plasticizers resemble the polymer structure and have the possible interaction capacity with the polymer. Shorter chain polymers (fewer monomers and overall lower Mw) can act as plasticizers. As used herein, short chain polymers refer to polymers having a Mw of less than 100,000 g/mol. Addition of plasticizer increases the energy necessary to initiate a crack in the coating. Plasticizer decreases Young's modulus and glass transition temperature of the specimen, effectively reducing the internal stress and decreasing the incidence of cracking.

In some embodiments it is desirable to use a coating material with a low melt flow temperature. Use of such coating materials may help to avoid coating process conditions (e.g., injection temperature and injection pressure) that could deform the core tablet. In some embodiments, the melt flow temperature of the coating composition is between about 70 □ and 200 □, or between 70 □ and 160 □. Other values are also possible.

Young's modulus, also called the elastic modulus, is estimated from the slope of the linear region of the stress-strain profile where the formulation experiences elastic deformation. This fundamental material property shows the elasticity of the film with lower values corresponding to higher elasticity. It evaluates the specimen resistance to the elastic deformation. The values are directly related to the interatomic bonding energy, with higher values corresponding to the stiffer and rigid film where it needs higher loads to deform elastically. In some embodiments, the coating polymers having low values of Young's modulus provide for better IM coating. The employed methodology differentiated between the coating formulations, considering their capability to resist the deformation. A low Young's modulus is advantageous in averting initiation and propagation of cracks, especially when the material has a higher strain. Cases when the material has a low Young's modulus because it deforms or breaks at a low stress is generally less desirable than a material with a similar Young's modulus having a higher strain value. In some embodiments, the coating composition has a Young's modulus of less than 700 MPa when measured at ambient conditions. In some embodiments, the coating composition has a Young's modulus of between 40 and 2200 MPa when measured at ambient temperatures.

Tensile strength is the maximum force per unit area applied to the specimen. It is the maximum stress that a specimen can withstand before necking or cracking. In some embodiments, it was found that a higher ratio of Young's modulus to tensile strength indicated higher resistance to coat cracking. The derived mechanical parameter, tensile strength/Young's modulus ratio indicates crack resistance and could predict cracking. Based upon the requirement of the tough and elastic nature of the tablet coat, coating formulations having a high ratio of tensile strength/Young's modulus would resist the external forces and stresses and have a lower tendency towards the cracking. PVA, hydroxypropyl pea starch, and Opadry based formulations had high values compared to other coating formulations and these coating formulations were the most successful in IM coating.

Toughness, the total area under the stress-strain curve, is a measurement of the energy absorption before failing. Toughness indicates material's resistance to breakage. Toughness of the specimen depends on both the strength and ductility of the specimen. Toughness was found to be a good indicator to look for in screening formulations for IM coating, as discussed in the Examples below. In some embodiments, coating compositions having toughness values higher than 95×10⁴ J/m³ (for example, Opadry, PVA, hydroxypropyl pea starch and PEO 1,000,000 based formulations, discussed further in the Examples below) performed well in IM coating. It was also found that in some embodiments, coating compositions having a melt flow of greater than 0.4 g/min and/or an elongation value of greater than 35% were found to be robust for IM coating.

The coating may have a range of thicknesses. In some embodiments, the target range of the coat thickness is less than 500 μm. In some embodiments the coating has an average thickness of between 150 and 300 microns. The coating thickness may be selected to provide a desired drug dissolution lag time. In some embodiments the coating thickness is selected to provide an increase of drug dissolution of between 10 and 35 minutes over that for uncoated tablet core material.

The following are examples of the invention. These examples are intended for illustration rather than limitation of the invention.

### EXAMPLES

### Example 1

An injection molding process was designed and tested for coating an immediate-release, injection-molded tablet core, designed to have an “immediate release” coat, with a dissolution lag time of not more than 15 minutes.

The tablet core used was the uncoated injection molded tablet. The core composition consisted of griseofulvin (GRIS, at 10% wt) as the drug in an excipient matrix of maltodextrin and xylitol. The tablet cores were prepared using a hot-mold extruder coupled to an injection molding machine.

The materials used included: Griseofulvin (GRIS) USP; Polyethylene oxide (Polyox N10, PEO); Xylitol (XYL) and polyethylene glycols (PEGs) 1500; Maltodextrin (MDX) (Glucidex IT 12); and Allura red dye.

Hot melt extrusion was performed in a co-rotating twin screw extruder (Leistritz Nano-16, America). The extruder had a top gravimetric feeder and four temperature zone barrel. The temperature of feed zone was maintained at 25° C. A 0.5 cm cylindrical exit die was used to obtain rod shaped extrudates.

An integrated set-up of hot melt twin-screw extruder (Leistritz Nano-16, America) coupled to a six cavity injection mold machine (MHS Hot Runner, Canada) was used for generating molded tablets. The mold machine halves opened in horizontal direction and had six tablet mold cavities. The powder blend feed rate to extruder was in range of 65-75 g/h with adjustments made during the operation based on mold cycle time.

An in-house built injection mold machine was used. The unit consisted of injection piston, temperature controlled injection barrel, temperature controlled mold cavity comprising two mold halves and orifice, and adjustable ejection pin. The unit has a process control and data acquisition system to control and monitor process parameters such as injection pressure, barrel temperature, mold temperature, hold time, etc.

The unit had one mold cavity that opened in the vertical direction. The injection barrel had two heating bands to maintain temperature of barrel and nozzle area.

The two mold halves were maintained at the desired temperatures. An adjustable ejector pin was installed for working with mold inserts of different depths. It was operated using Tritex and LabVIEW software. The process parameter of injection speed was controlled by Tritex software. The LabVIEW program was used to set process parameters of injection pressure, injection time, hold pressure and hold time. Real time injection pressure-time profiles were recorded.

The extrusion-molded tablet cores were characterized for dimensions. The tablet had asymmetrical halves and had a radius of curvature. Tablet images captured on an optical comparator were imported in to a LabVIEW program to obtain tablet drawings. Coat mold inserts with target coat wall thickness were then designed to complement the uncoated tablet shape.

TABLE 1
Coat dimensions for coat mold sets used in the study.

| | | Coat thickness at | |
|---|---|---|---|
| | Coat thickness | weld | |
| Coat mold design | (micron) | (micron) | Weld type |
| Set 1 | 150 | 350 | Overlap at weld |
| Set 2 | 300 | 500 | Overlap at weld |
| Set 3 | 300 | 300 | No overlap at |
| | | | weld |

The coating process steps were similar for all coating trials, and involved a multi-step process. Step 1 comprised coating the ‘pot’ or lower half of uncoated tablet. After coating, the sprue attached to the coated side was manually cut off. The half coated tablet was then inverted and placed in STEP 2 mold inserts and the ‘lid’ or top half was coated. After the tablet ejected, the sprue was manually cut off.

PEO plasticizer composition (Table 2) was aimed to achieve the following (a) a fast dissolving coat of PEO N10, (b) lower the melt viscosity of PEO N10 composition such that the tablet coating process parameters of injection temperature and injection pressure can be lowered to avoid deformation of the tablet core, during the coating process and (c) yield homogenous PEO-plasticizer matrix to achieve acceptable coat film properties. Low molecular weight PEG 1500 was added as plasticizer for PEO N10.

TABLE 2
Coat composition prepared by hot melt extrusion.

| Ingredients | Formulation (% wt) |
|---|---|
| PEO N10 | 71.5 |
| PEG 1500 | 28.5 |
| Hot melt extrusion temperature (° C.) | 100 |

* Altura red dye was added to coat composition for visual differentiation between tablet and coat.

The extrusion molded tablets had asymmetrical halves. The greater thickness half (2.85 mm) was taken for first half coating (STEP 1). Coated tablets were characterized for appearance, weight gain, tablet thickness and coat thickness uniformity.

TABLE 3
Coating process parameters for coat trial with SET 2 molds.

| | 1^{st} half coat | 2^{nd} half coat |
|---|---|---|
| Process parameters | STEP 1 | STEP 2 |
| Injection pressure (psi) | 4000 | 20,000-27,000 |
| Injection time (s) | 2.5 | 4 |
| Hold pressure (psi) | 4000 | 25,000 |
| Hold time (s) | 2.5 | 3 |
| Barrel temperature (° C.) | 90 | 90 |
| Mold temperature (° C.) | 30 | 30 |
| Solidification time (s) | 5 | 5 |
| Injection speed (cm/s) | 0.845-4.23 | 4.23 |

Coating STEP 1—

The PEO coat formulation had optimal melt flow behavior and a full coating could be achieved at the low injection pressure of 4000 psi. At injection speed of 8.45 cm/s, the overshoot was NMT 7000 psi. However, when the injection speed was increased to 4.23 cm/s, due to the limitation of in-house instrument, the injection pressure showed overshoot up to about 14,000 psi and then reduced to plateau at 4000 psi.

Coating STEP 2—

The STEP 2 of the process involved coating of the ‘lid’ half of the tablet as well as the weld formation. Optimal coat thickness and weld was obtained in the injection pressure range of 20,000-25,000 psi.

Coated tablets were examined for weight and dimensions. Tablet thickness and diameter were recorded using micrometer (Mitutoyo, Japan) with 1 micron resolution.

TABLE 4
Measurements of coated tablet dimensions and weight uptake for 300
micron coat (500 micron at weld).

| | | 1^{st} half coated | 2^{nd} half coated |
|---|---|---|---|
| Parameters | Uncoated tablet | tablet | tablet |
| Weight (mg) | 522.7 ± 6.4 | 596.8 ± 2.4 | 637.1 ± 1.6 |
| (% wt gain) | — | (13.6 ± 0.4) | (7.1 ± 0.4) |
| Tablet thickness | 5.749 ± 0.010 | 6.032 ± 0.007 | 6.322 ± 0.018 |
| (mm) | | | |
| Tablet diameter | 9.95 ± 0.010 | 10.623 ± 0.015 | 11.021 ± 0.010 |
| (mm) | | | |

The tablet coat thickness and weld area examination was performed using optical microscope at 20× magnifications. Measurements were made using an Infinity Analyzer v.5 software. Samples were also examined by using SEM-samples were gold sputter coated before analysis.

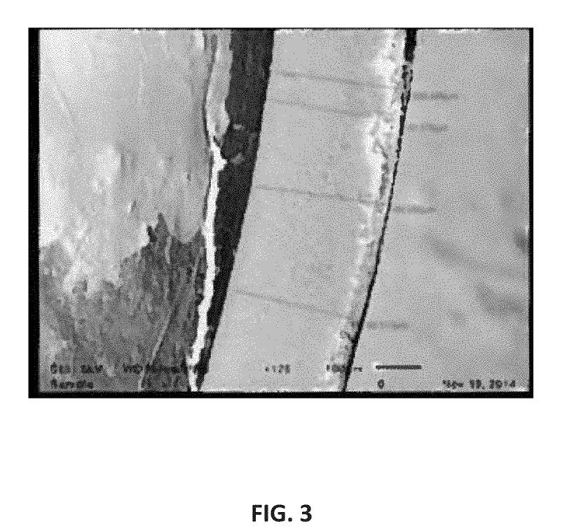

For half coated tablets, the coat thickness uniformity was inspected (FIG. 3). The measurements were corroborated with SEM measurements. The half coated tablets were then taken for the next step of coating. FIG. 3 shows optical microscope (20×) and SEM images of half coated tablet for coat thickness measurements.

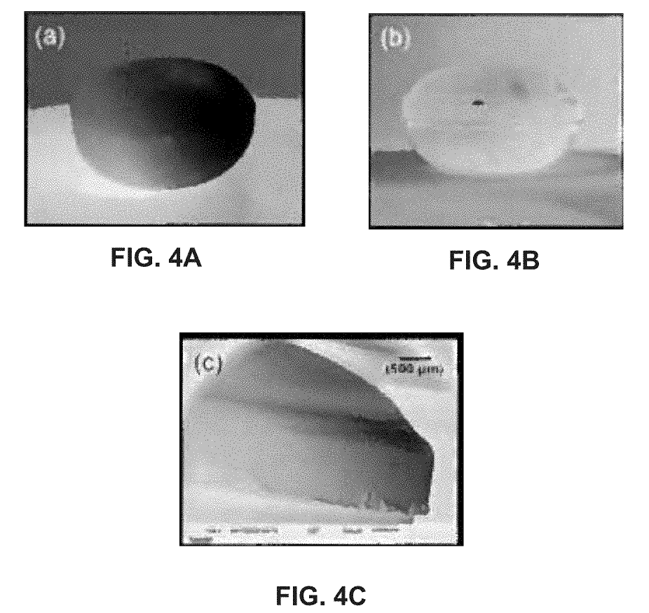

FIGS. 4A and B show uncoated and coated tablets, respectively. FIG. 4C shows an SEM of the weld area, indicating a smooth coat and no exposed core. The second-half coating process conditions were optimized for weld behavior. The injection pressure and barrel temperature were critical process parameters that effected weld behavior.

### 4.5.3 In Vitro Drug Release Performance

In vitro drug release testing was performed using United States Pharmacopoeia paddle (Apparatus 2) on a Agilent 708-DS dissolution tester, (Agilent Technologies, Santa Clara, Calif.). The dissolution medium was 1000 ml of aqueous sodium lauryl sulphate solution (40 mg/ml) at temperature of 37° C. The rotational speed of the paddles was set at 75 rpm. Samples were analyzed by UV spectrophotometer at 291 nm wavelength.

GRIS release from uncoated tablets was ˜80% in 15 min, thus demonstrating the potential advantage of MDX matrix for fast dissolving tablets. The 150 and 300 micron coated tablets, with PEO composition, showed lag times of about 5 and 10 min, respectively for start of drug release. The dissolution lag time to achieve 80% of drug release was about 5 and 15 min, respectively, as compared to the time taken for the uncoated tablets.

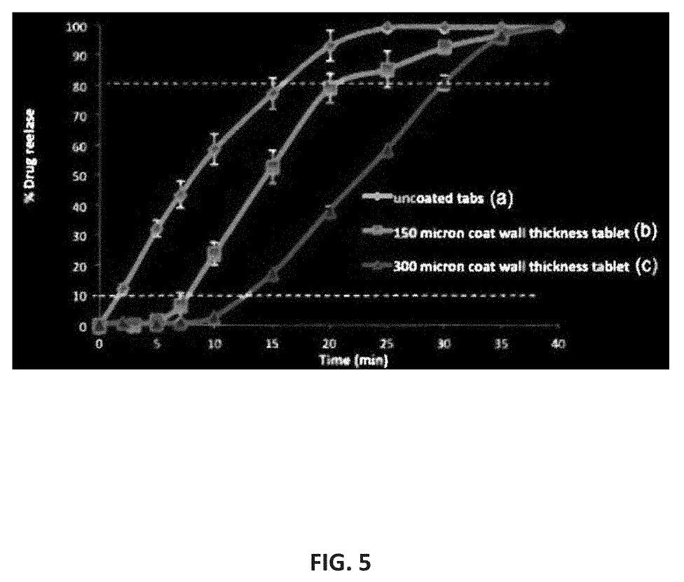

FIG. 5 shows dissolution profiles for (a) uncoated injection molded tablet, (b) coated tablets with coat 150 micron, overlapping region 350 micron (SET 2) and (c) coated tablets with 300 micron, overlapping region 500 micron (SET 1) (n=3, mean±SD).

### Example 2

Testing was performed to provide injection-molded coatings on tablets. An MIT built vertical injection molding unit was utilized to coat the tablets. The unit comprised injection piston, temperature-controlled injection barrel, temperature-controlled mold cavity comprising two mold halves and orifice, and adjustable ejection pin. The unit included a process control and data acquisition system to control and monitor process parameters. Injection pressure, barrel temperature, mold temperature and hold time were the major process parameters controlled during the study. Briefly, the tablet was placed into the mold cavity and the injection barrel was filled with coating material. The tablet was coated in two steps with a coating thickness of 300 μm. An injection piston at a particular pressure was used to introduce the plasticized coating material into the mold cavity. Material solidified at the lower mold temperature. The tablet core was placed in the mold cavity and a first half of the surface of the tablet core was coated. The tablet was the inverted and the second half of the tablet surface was coated by the same process.

Two different types of tablets were used in the study, injection molded griseofulvin tablets and machined polyetherimide tablets. Coating material was mainly a combination of coating polymers and plasticizers in different concentrations. Coating polymers used in the study were polyethylene oxide, acrylates, polyvinyl alcohol, polyvinyl alcohol/polyethylene glycol graft copolymer, polyethylene glycol, polyvinyl acetate, and polyvinylcaprolactame-based graft copolymer. Glycerine and polyethylene glycol were used as plasticizers.

Modulated differential scanning calorimetry (MDSC) was used to study glass transition temperature and thermal properties of all the materials. Stress strain analysis of coating formulations was accomplished using a universal testing machine. Melt rheology of coating formulations was studied using a rheometer. Tablets were coated with selected coating formulations and stored in different temperature and humidity conditions for long-term stability testing. Coat stability over time was analyzed by stereo-microscopy to identify the suitable coating formulation for injection molding and to confirm the applicability of injection molding coating technique for tablet coating. In vitro dissolution performance was performed to check the suitability of coating material for immediate release and controlled release formulations.

Polyvinyl alcohol based coating formulations had lower Young's moduli and higher ratios of Young's modulus to tensile strength (indicating higher resistance to coat cracking) in comparison with acrylate and polyethylene oxide based formulations. On the other hand, MDSC results confirmed that polyvinyl alcohol based formulations had higher glass transition and melting point temperatures, suggesting that the polyvinyl alcohol based formulations may be preferably manufactured through injection molding coating processes at injection temperatures greater than 130° C. Tablets were successfully coated using different coating formulations by two steps injection molding process. In conclusion, injection molding may serve as a promising alternative technology for tablet coating.

### Example 3

This Example explores the application of HME-IM (hot melt extrusion/injection molding) process technology for development of a model pharmaceutical coated tablet i.e. a “core-coat” formulation system. The selected “core” tablet was an extrusion molded tablet. The tablet formulation comprised maltodextrin-xylitol matrix with 10% w/w griseofulvin (GRIS) and were prepared by integrated HME-IM process. The aim of the study was to develop tablet coating process using IM process technology. The specific aims of the study were:
(i) develop and demonstrate IM process for coating of extrusion molded tablets with target coat thickness of less than 500 micron
(ii) develop an immediate release coat formulation suitable for IM tablet coating process, and
(iii) demonstrate acceptable coat morphological properties and a seal at the weld.

### Materials

GRIS (USP, particle size <10 μm) was purchased from Jinlan Pharm-Drugs Technology Co. Limited. (Hangzhou, China). Maltodextrin (MDX, glucidex IT 12 grade), and xylitol (XYL, Xylisorb® 90) were kindly provided by Roquette America Inc. (Geneva, Ill.). Polyethylene oxide (Polyox N10, PEO) was obtained as a gift sample from Dow Chemical Company (USA). Polyethylene glycols (PEG) molecular weights 400, 1500, and 35,000, and allura red dye were purchased from Sigma (St. Louis, USA).

### Methods

### Generation of ‘Core’ Tablets by Integrated Hot Melt Extrusion-Injection Molding Process

A set-up of a hot melt twin screw extruder (Leistritz Nano-16) coupled to a horizontally opening injection mold machine (MHS Hot Runner, Ontario, Canada) was used for generating tablets. The major parts of the IM machine were a heated reservoir, and a hot runner system (Rheo-Pro® Hot Runner Systems) which comprised of a manifold, injection nozzle, and six valve gates that led to six tablet mold cavities.

### Preparation of Coat Formulation by Hot Melt Extrusion

HME was performed in a co-rotating twin-screw extruder (Leistritz Nano-16, Somerset, N.J.). The extruder barrel has four heating zones and a 0.6 cm cylindrical exit die. The feed zone temperature was maintained at 25° C. For all formulation trials, accurately weighed ingredients were screened through a 600 μm pore size sieve and blended for 10 min in a Turbula mixer (GlenMills Inc., Clifton, N.J.). The pre-mixed powder blend was fed into the extruder by a top gravimetric feeder. Allura red dye (company) was added to the coat formulations.

### Tablet Coating on Vertical Injection Molding Machine

### Description of Vertical Injection Molding Machine and Mold Inserts

An MIT in-house built vertical injection mold machine comprising of one mold cavity was used. The injection barrel had two heating bands to maintain temperature of the barrel and nozzle area. Mold bases were fixed on the top and bottom platens of the machine. Interchangeable mold inserts were used for the different coating stages. The top and bottom mold halves were maintained at the desired temperature using circulating fluid. An adjustable ejector pin was installed for working with mold inserts of different depths. The machine operation including the process parameter of injection speed was controlled using Tritex software. The coating material (i.e. either “as is” powder or HME extrudates) was transferred into the injection barrel and allowed to melt at the set barrel temperature.

### 3.3.3 Description of LabVIEW Program Used to Control and Record Injection Molding Coating Process Variables-Time Profiles.

The IM process parameters of injection pressure, injection time, hold pressure, and hold time were controlled with a LabVIEW program. The program also supported recording of real-time injection pressure profiles.

The objective was to design a control algorithm to track a given pressure profile (set point) for the IM process. The control system should be able to track the pressure set point for both the filling and packing stages in the IM process, with specific performance criteria being low overshoot and fast setting time in the presence of process disturbances.

A two-level control strategy was designed that switches between open- and closed-loop control depending on the value of the measured pressure

The controller strategy is implemented using LabVIEW software. A load cell is mounted at the bottom of the mold to measure the force of the injection. The force measurement is fed back to the LabVIEW program which first converts the force into the equivalent pressure. Then Algorithm 1 compares the measured pressure with a given setpoint, and determines the necessary control move. Finally, the calculated controller move is implemented by sending a command to the process driver.

### Characterization of Tablet Coat

Molded circular disks of coating compositions and coated tablets were examined for physical appearance, weight and dimensions. For the circular disks, thickness was monitored, and for the coated tablets thickness and diameter were monitored using micrometer (Mitoyto, Japan) with least count of 0.01 mm. Tablet dimensions and weight measurements were performed on samples immediately after ejection.

Tablet coat thickness and weld area were examined using an optical microscope at 20 and 50× magnifications and Infinity Analyzer v.5 software. Samples were also examined by scanning electron microscope (SEM) using Jeol-6060 (Tokyo, Japan). SEM samples were gold sputter coated before analysis.

Dissolution testing of tablets was performed as per United States Pharmacopoeia (USP) method Test 1 for GRIS tablets (USP 37-NF 32) using a USP apparatus II (paddle) dissolution tester (Agilent 708-DS, Agilent Technologies, Santa Clara, Calif.). The dissolution medium was 1000 ml of aqueous sodium lauryl sulphate solution (40 mg/ml) maintained at a temperature of 37±0.5° C. The rotational speed of the paddles was set at 75 rpm. Samples of 5 ml were withdrawn at specific time points, filtered through 0.22 μm nylon filter, appropriately diluted, and analyzed by UV spectrophotometer at a wavelength of 291 nm. Each formulation was tested in triplicate.

### Results and Discussion

### Injection Molding Tablet Coating Process Development

To coat tablets by the IM process, several strategies were explored. We selected a two-step the tablet coating molding process like that illustrated in FIGS. 1A-1C discussed above. The ‘core’ extrusion molded tablets had asymmetrical halves and a parting line. The appearance of the tablet was similar to a cooking pot, therefore, the top shallow half was referred as the ‘lid’ and the bottom deeper half, with greater height, was referred to as the ‘pot’. The Step 1 of the process comprised coating the ‘pot’ half of the core tablet. Following this, the partially coated tablet was ejected and the sprue attached to the coated side was manually cut. In the Step 2, mold inserts were changed and the half-coated tablet was inverted and placed in the Step 2 mold inserts and the ‘lid’ half of the tablet was coated. After tablet ejection from the mold the sprue was manually cut.

To develop the coat mold tooling, the first step was to accurately characterize the dimensions of the ‘core’ extrusion-molded tablets. In this study, we selected the model ‘core’ tablet made by extrusion molded process, as we were interested in evaluating the IM process technology for tablet manufacture and coating operation. However, ‘core’ tablets prepared by other process technologies such as powder compression could also be viable options. The extrusion molded ‘core’ tablet had asymmetrical halves and had varying radius of curvature, therefore the tablet images captured on an optical comparator and imported into a LabVIEW program to obtain tablet drawings and develop an offset geometry of the coated tablet (FIG. 3). Based on this, coating mold inserts for the target coat thickness were designed to complement the ‘core’ tablet shape.

Three coating molds design were evaluated in this study, namely (i) 300 μm coat mold with no overlap at the weld (Set 1), (ii) 300 μm coat mold with overlap at the weld area (Set 2) and (iii) 150 μm coat mold set with overlap at the weld area (Set 3). Table 5 shows coat dimensions used in this study. Each set of coating molds comprised 3 mold inserts. The coating process steps used were similar for the three coat designs.

TABLE 5
Summary description of the three coat mold designs.

| | Coat | Thickness at | |
|---|---|---|---|
| Mold | thickness | weld | |
| design | (μm) | (μm) | Weld type |
| Set 1 | 300 | 300 | Non-overlapping |
| Set 2 | 300 | 500 | With overlap |
| Set 3 | 150 | 350 | With overlap |

### Rationale for Selection of ‘Core-Coat’ System Composition

The target range of the coat thickness was less than 500 μm with drug dissolution lag time in the range observed for conventional immediate release coated formulations (i.e. meeting the in vitro release specification in the USP for griseofulvin tablets). With regard to processability, an important parameter was that the core tablet should be able to endure the coating process. The coating process conditions of injection temperature and injection pressure should not deform the core tablet. Therefore, it was desirable to use a coating material with a low melt flow temperature and melt viscosity. PEG and PEO, water-soluble crystalline polymers, with T. of about 66° C. were selected as the coating materials.

PEGs were selected due to their low melt flow temperature and melt viscosity, however, PEGs have brittle characteristics therefore a range of molecular weights (PEG 8000 to 35,000 molecular weight) were screened for their coat film properties by the IM process. Secondly, PEO N10, a high molecular weight PEG (100,000 Daltons) was selected as it has been shown to be a good film former and is processesable into melt casted films at the temperature of about 110° C. The PEO formulation was optimized for plasticizer type and concentration.

### Optimization of Tablet Coat Compositions

(i) Tablet coating trials with neat PEGs of 8,000, 20,000, and 35,000 molecular weights were performed on core tablets. It was observed that the PEG 8000 and 20,000 coat showed tendency of crack formation immediately after coating. However, distinctly the coat cracking behavior reduced with increase in molecular weight to PEG 35,000 and the cracks were observed to develop at later time points (stored at room conditions, 1 week). Therefore, PEG 35,000 coat composition was modified with partial addition of PEO N10 (Formulation F1 in Table 6) and was prepared by HME process to attain a uniform matrix.

(ii) The PEO film coat composition was optimized for plasticizer type and concentration. The plasticizer selection and optimization for PEO N10 was aimed to achieve the following (a) a fast dissolving coat of PEO N10, (b) lower the melt viscosity of PEO N10 composition such that the tablet coating process parameters of injection temperature and injection pressure can be lowered, and (c) yield homogenous PEO N10-plasticizer matrix to achieve acceptable coat film properties. The targeted upper limit for injection temperature was about 95° C. Low molecular weight PEGs, PEG 400 and 1500, were evaluated as plasticizers. Maximization of low molecular weight PEGs is expected to reduce the dissolving time of PEO N10 matrix as well as improve the molten mass flow behavior and therefore facilitate the coating process.

The PEO-PEG powder blends were first extruded at extrusion temperature of 95° C. and screw speed of 90 rpm to achieve homogenous extrudes. It was observed that at the lower extrusion temperature of 75° C., the obtained extrudates showed blooming after short duration of storage under room conditions. An extrusion temperature of 95° C. was found to yield optimal PEO-PEG extrudates with long-term stability. Formulation F2, thin film extrudate with PEG 400 at 23% w/w (i.e. 30% w/w of PEN N10 polymer) showed signs of blooming, and PEG phase separation was seen within one day of storage in glass vials at room conditions. As compared, Formulation F3 thin film extrudates containing PEG 1500 at 28.5% w/w concentration (i.e. 40% w/w of PEO N10 polymer) were homogenous and showed no signs of blooming when stored in aluminum sealed packs and eon exposure to open 25° C./55% RH conditions for 2 weeks.

Formulation F1 and F3 were feasible coat formulations, from which formulation F3 was taken forward for detailed assessment of the IM coating process trials. Flat circular disc of 300 μm were prepared by injection molding process to evaluate expansion/shrinkage behavior. Discs were prepared at an injection temperature of 90° C. and injection pressure of 4000 psi. Dimension monitoring of these discs showed no significant change in disc thickness for 21 days storage under room storage conditions (in closed glass vials).

TABLE 6
Composition of the coat formulations prepared by hot melt extrusion.

| | Formulation (% w/w) | | |
|---|---|---|---|
| Ingredients | F1 | F2 | F3 |
| PEO N10 | 21.4 | 77.0 | 71.5 |
| PEG 400 | — | 23.0 | — |
| PEG 1500 | 5.0 | — | 28.5 |
| PEG 35,000 | 73.6 | — | — |
| Extrudate | no blooming | shows | no blooming |
| appearance | | blooming | |

### Description of the 1^{st} Half Tablet Coating (Step 1)

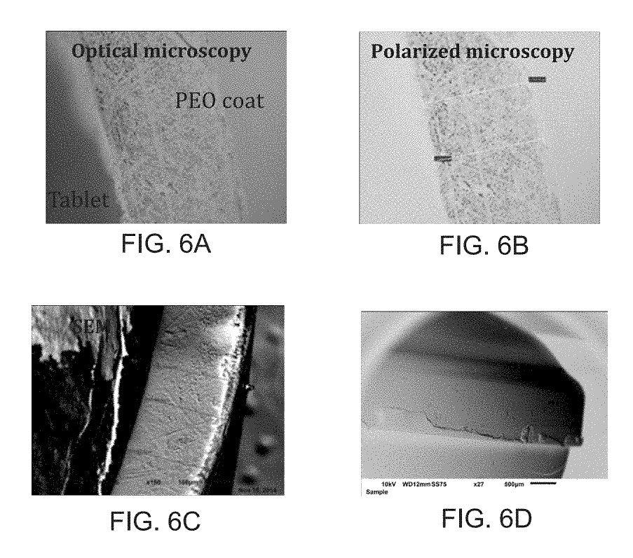

After coating the lower part of the tablets, the half-coated tablets were characterized for appearance, weight gain, tablet thickness and coat thickness uniformity. Table 4 lists the measurements and images of the half-coated tablets produced using the different set molds. The bottom half mold was same for the non-overlapping and overlapping 300 μm coat molds. FIGS. 6a and 6b show optical and polarized microscopy images. The optical microscope measurements were corroborated with SEM measurements (FIG. 6c). For each tablet, the coat thickness was measured at four points, at 90 degrees each. Half-coated tablets with coat thickness in range of 300±50 μm were taken for the step 2 i.e. the second “lid” half coating. A mild flash was observed in the weld area, which sometimes obstructed the observation by optical microscopy. This flashing would likely not be hard to eliminate using harder and higher quality steel tooling manufactured to tighter specifications. The cost of this endeavor was beyond the scope of this work.

Effect of Injection Pressure:

The PEO N 10 based, F3 formulation could yield a complete coat at injection pressure of 2000 psi. At an injection temperature of 90° C., an injection pressure of 2000 to 6000 psi yielded similar weight uptake and tablet dimensions. An injection pressure of 4000 psi was used further in all studies (Table 7).

Effect of Injection Speed:

At an injection speed of 0.845 cm/s the injection pressure overshoot was in range of 7000 psi. However, when the injection speed was increased to 4.23 cm/s, due to the limitation of the in-house IM instrument, the injection pressure showed overshoot up to about 14,000 psi and then reduced to plateau at 4000 psi. The coating was found to be complete at both speeds and no core tablet deformation was observed.

TABLE 7
IM process parameters for step 1 and 2 of the tablet coating process,
using the Set 2 molds.

| | Step 1 | Step 2 |
|---|---|---|
| Process parameters | 1st half coat | 2nd half coat |
| Injection pressure (psi) | 4000* | 20,000-25,000 |
| Injection time (s) | 2.5 | 4 |
| Injection hold pressure (psi) | 4000 | 25,000 |
| Injection hold time (s) | 2.5 | 3 |
| Molten mass temperature | 90 | 90 |
| (° C.) | | |
| Mold temperature (° C.) | 30 | 30 |
| Solidification time (s) | 5 | 5 |
| Injection speed (cm/s) | 0.845, 4.23 | 4.23 |
| Margin | 0.3 | 0.7 |
| Current limit (Amp) | 1.5 | 5 |

*There was an initial pressure spike as described in the text.

### Description of the 2^{nd} Half Tablet Coating (Step 2)

The step 2 of the coating process involved coating the “lid” portion as well as the weld formation with the first half-coat on the tablet. The injection pressure, injection speed and barrel temperature were critical process parameters that effected weld formation and efficacy. In this case, since a fast injection speed would promote a better weld, therefore the maximum speed of 4.23 cm/s was used.

At an injection temperature of 90° C., an injection pressure of less than 10,000 psi showed a failure to seal the 2 coating parts at the weld line. When the molds opened for tablet ejection, the formed lid′ coat stayed attached to the top mold and did not adhere to the tablet. At injection pressure in range of 16,000 psi, the second half coat was achieved, however the weld area showed regions were the two coat halves did not meet and the uncoated tablet was exposed. On further increase of the injection pressure to the range of 20,000-25,000 psi, the coated tablet thickness increased to 6.3 mm (i.e. closer to the nominal mold dimensions). The IM process parameters used for further coating studies are listed in Table 3.

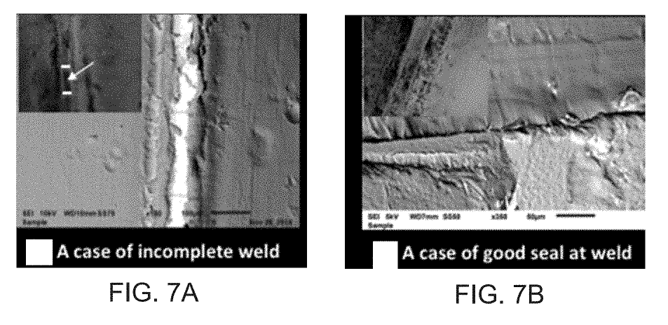

At the established IM parameters, trial conducting using non-overlapping 300 μm coat molds (Set 1), showed successful weld, however the probability of variation was higher and in some cases a gap in the weld region was observed when examined under microscope and by SEM (FIG. 7). With use of the overlapping 300 μm coat molds (Set 2), the produced weld showed overlap of the two coat halves throughout the tablet diameter and was consistent for the multiple trials. Weld area of the full-coated tablet (using Set 2 molds), was inspected by optical microscopy and SEM and found to be smooth with no crack observation (FIG. 6d).

Tablet Coating Trial with the Overlapping 150 μm Coat Molds (Set 3):

The established IM process parameters were used to successfully achieve coated tablets with 150 μm coat thickness, using the Set 3 molds. The coat was visually thinner, and a complete seal was observed at the weld region. For formulation F3, the total % weight gain for tablet coating of 300 μm and 150 μm thickness was about 22% and 16% w/w, respectively. Further, coated tablets were prepared with the PEG based coat (formulation F1) at 300 and 150 μm coat thickness, using the same IM process parameters.

FIG. 6 shows microscopic and SEM images of weld region of coated tablet using 300 μm coat mold with no overlap at weld (Set 1) (arrow shows gap in between two coat halves), using coat formulation F3. FIG. 7A is a case of an incomplete weld. FIG. 7B is a case of a good seal at weld.

### Evaluation of In Vitro Drug Release of Coated Tablets

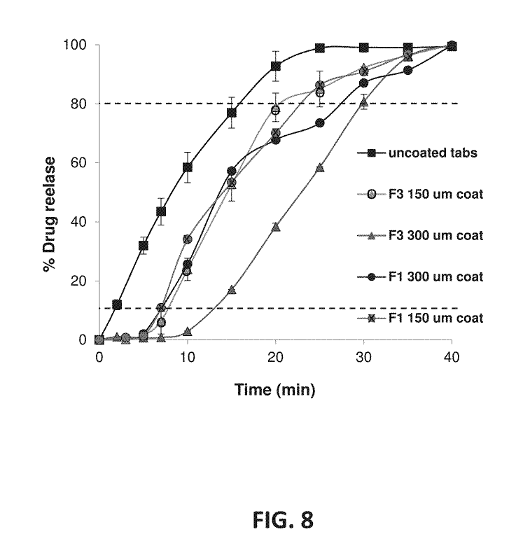

The impact of tablet coat on dissolution performance was assessed for the different coat compositions and different coat thickness (FIG. 8). GRIS dissolution from the core tablets was about 12% in 2 min and 80% in 15 min. For the PEO based F3 formulation coat, the 150 and 300 micron coated tablets showed lag times of about 5 and 10 min, respectively, for the start of drug release. In reference to the core tablet, the lag time to attain 80% of drug release was about 5 and 15 min, respectively. For the PEG based F1 formulation coated tablets, the coat wall thickness of 150 and 300 μm demonstrated a similar lag time (about 5 min) and drug release profiles. As compared to PEO N10, PEG is a smaller molecular weight fast dissolving material, and was expected to show less impact of coat thickness on lag time. FIG. 8 is a graph showing GRIS dissolution profiles for core extrusion molded tablets and coated tablets using coat formulation F1 and F3 at coat thickness of 150 and 300 micron (n=3, mean±SD).

### Conclusions

The study demonstrates use of an IM process technology for full continuous coating (no substantial holes or openings) on a pharmaceutical tablet. The developed IM process and tablet coats achieved acceptable appearance, a viable seal at the weld region, and also achieved the desired fast drug release performance. The variations in mold design and coat thickness provided useful understanding of the coat thickness range and corresponding weight uptake on tablets, for comparing with conventional tablet coating process.

### Example 4

In this Example, a solvent-free injection molding (IM) coating technology was developed that could be suitable for continuous manufacturing via incorporation with IM tableting. Coating formulations (coating polymers and plasticizers) were prepared using hot-melt extrusion and screened via stress-strain analysis employing a universal testing machine. Selected coating formulations were studied for their melt flow characteristics. Tablets were coated using a vertical injection molding unit. Process parameters like softening temperature, injection pressure, and cooling temperature played a very important role in IM coating processing. IM coating employing polyethylene oxide (PEO) based formulations required sufficient room humidity (>30% RH) to avoid immediate cracks, whereas other formulations were insensitive to the room humidity. Tested formulations based on Eudrajit E PO and Kollicoat IR had unsuitable mechanical properties. Three coating formulations based on hydroxypropyl pea starch, PEO 1,000,0000 and Opadry (PVA-based) had favorable mechanical (<700 MPa Young's modulus, >30% elongation, >95×10⁴ J/m³ toughness) and melt flow (>0.4 g/min) characteristics, that rendered acceptable IM coats. These three formulations increased the dissolution time by 10, 15 and 35 minutes respectively (75% drug release) compare to the uncoated tablets (15 minutes). Coated tablets stored in several environmental conditions remained stable to cracking for the evaluated 4-week time period.

### 1. Introduction

To achieve the described continuous coated tablet manufacturing, IM coating is required to be thoroughly analyzed first as a separate technology by evaluating coating formulation attributes and IM process parameters. IM coating technology has not been explored in detail.

### 2. Materials and Methods

### 2.1 Materials

Injection molded core griseofulvin (GF) tablets were formulated from Griseofulvin USP (Jinlan Pharm-Drugs Technology Co. Limited., Hangzhou, China), maltodextrin (Glucidex IT 12, Roquette America Inc. Geneva, Ill.), xylitol (Xylisorb® 90, Roquette America Inc., Geneva, Ill.) and anhydrous lactose (SuperTab 24AN, DFE Pharma, Paramus, N.J.). Custom shaped polyetherimide (Ultem™ 1000, PEI) tablets were purchased from Proto labs (Maple Plain, Minn.). A wide variety of coating polymers were employed to coat these tablets and are listed here. Polyethylene oxide [PEO 100,000 (Polyox WSR N-10), PEO 300,000 (Polyox WSR N-750), PEO 1,000,000 (Polyox WSR N-12K)] were obtained from the Dow Chemical Company (Midland, Mich.). Polyvinyl alcohol (PVA, Gohsenol™ EG-05 PW) was received from Nippon Gohsei (Osaka, Japan). Amino Methacrylate Copolymer-NF (Eudragit E PO) was acquired from Evonik (Darmstadt, Germany). Polyvinyl alcohol-polyethylene glycol graft copolymer, Kollicoat IR (Kollicoat) was procured from BASF (Ludwigshafen, Germany). Polyvinyl alcohol based copolymer, Opadry 200 (Opadry) was acquired from Colorcon (Harleysville, Pa.). Hydroxypropyl pea starch (Readylycoat) was received from Roquette (Keokuk, Iowa). The plasticizers polyethylene glycol (PEG 400, PEG 1500) and glycerol were purchased from Sigma-Aldrich (St. Louis, Mo.), whereas acrylate based plasticizer (Eudrajit NE 30D) was obtained from Evonik (Darmstadt, Germany). Potassium acetate, magnesium chloride, potassium carbonate and magnesium nitrate salts were purchased from Sigma-Aldrich (St. Louis, Mo.). Propylene glycol-water mixture (Dowtherm SR-1 35, The Dow Chemical Company, Midland, Mich.) was used as a coolant.

### 2.2 Methods

### 2.2.1 GF Tablet Manufacturing

An integrated HME-IM continuous tablet manufacturing platform was used to manufacture GF tablets. Formulation constituents, GF (drug), xylitol (plasticizer) and lactose (reinforcing agent) were used as received, whereas maltodextrin (polymer carrier) was dried to achieve the residual moisture less than 0.5%. Briefly, premixed blend of GF (10%), dried maltodextrin (54.4%), xylitol (32.6%) and lactose (3%) were fed through weight-in-loss feeder to the feed zone of the co-rotating intermeshing twin screw extruder (Nano 16, Leistritz, Somerville, N.J., USA). The feed flow rate was 80 g/hr, whereas the screw speed was maintained at 90 rpm and feed zone temperature was 8° C. Formulation ingredients were mixed and sheared at elevated temperatures progressing with zone temperatures of 80° C., 155° C., 155° C. and 155° C. inside the extruder barrel. The melt extrudate, coming out from the extruder, was directly fed to the reservoir of the attached IM unit (MHS Hot Runner Solutions, Ontario, Canada). The IM unit could be divided into reservoir system and hot runner system (comprised of manifold and nozzles). The reservoir, manifold and nozzle temperatures were maintained at 150° C., 145° C. and 135° C., respectively. The extrudate progressed from the reservoir and hot runner systems to the mold cavities of the IM system and got solidified at 45° C. for 30 s to form core IM GF tablets. Since the melting point of GF is very high (˜220° C.), the employed processing conditions and polymer carrier maintained the stable crystalline nature of GF in the core IM tablets. The crystalline nature of the griseofulvin in tablet matrix was confirmed by X-ray diffraction analysis (data not shown).

### 2.2.2 PEI Tablet Manufacturing

Computer-aided design (CAD) model of the tablet was provided to Proto Labs. This custom prototype manufacturer employed Computer Numeric Control (CNC) milling process to manufacture the required precise shaped PEI tablets having 10 mm diameter and 5.7 mm maximum thickness at the center.

### 2.2.3 Preparation of Coating Formulations

TABLE 8
Coating formulations and processing temperature used for their preparations

| | | Extrusion Processing |
|---|---|---|
| Coating polymer | Plasticizer (% w/w) | temperature (° C.) |
| PEO 100,000 | PEG 1500 (10%, 20%, 30%) | 90, 80, 80 |
| PEO 300,000 | PEG 1500 (10%, 20%, 30%) | 120, 115, 95 |
| PEO 1,000,000 | PEG 1500 (10%, 20%, 30%) | 95, 95, 95 |
| PVA | Glycerol (20%, 30%, 40%) | 170, 170, 170 |
| PVA | PEG 400 (10%, 20%, 30%) | 180, 180, 180 |
| PVA | PEG 1500 (20%) | 190 |
| Eudragit E PO | Eudragit NE 30D (10%, 20%, 30%, 40%) | 100, 100, 100, 100 |
| Kollicoat | PEG 400 (10%, 20%, 30%) | 185, 170, 150 |
| Kollicoat | PEG 1500 (20%) | 180 |
| Kollicoat | Glycerol (20%) | 170 |
| Opadry | Glycerol (20%, 25%, 30%) | 165, 155, 150 |
| Hydroxypropyl pea starch | Glycerol (30%) | 100 |

Different ratios of plasticizer employed in the trials are provided in each row by providing its percentage value. Processing temperature values correspond to these polymer-percentage plasticizer combinations in same sequence (i.e., PEO 100,000+10% PEG 1500 coating formulation was processed at 90° C.; PEO 100,000+20% PEG 1500 coating formulation was processed at 80° C. and so on).

A vertical, co-rotating conical, miniature, twin-screw extruder (DACA instruments, Goleta, Calif.) was employed to prepare coating formulations. The screws with 14.5 mm diameter at the entrance and 5.5 mm at exit were enclosed in a heated jacket having an exit port. Coating polymer and plasticizer in particular ratios were weighed, premixed and fed to the extruder through the feed port. The amount of this mixture (3-5 grams) was determined depending on the torque and the volume occupied in the extruder. The screw speed was set at 100 rpm for all coating formulations. The unique design of this extruder with a featured recirculation channel allowed recirculation and thorough mixing of polymer mixtures inside the extruder. After recirculating for 5 minutes, the output valve was opened and the extrudate was collected through the exit port. Extrudates having a well-mixed appearance and no scaling were chosen for further study. Extrusion was first tried at extruder temperatures near the polymer glass transition temperature and/or melting temperature reported in literature. Later, they were optimized depending upon the polymer-plasticizer combination and extrudate characteristics (Table 8). The extruder temperatures that provided extrudates without any visual phase separation, and scaling were used to prepare coating formulations.

### 2.2.4 Tensile Testing

Specimens for the tensile testing of coating formulations were produced using a microinjector (DACA instruments, Goleta, Calif.). The instrument consists of a heated block that supports the conical, self-clamping mold and a heated barrel. The coating formulation extrudates obtained from the miniature twin-screw extruder were cut into small pieces. The barrel was then manually filled with these extrudate pieces. An injection piston, pneumatically driven by a bore cylinder forced the coating formulation from the barrel into the dogbone shaped mold cavity. As a starting point, temperature required to extrudate the coating formulation from the miniature twin-screw extruder was used as the barrel temperature. Then, the barrel temperature was further optimized (typically increased) to achieve a fully filled mold cavity at the selected barrel temperature. The mold temperature was maintained at 35° C. for all coating formulations. In the results and discussion section, the optimized barrel temperature values required for the specimen preparations of each coating formulation are provided along with their tensile properties. The length, width and thickness of the test regions of the prepared specimens are 25 mm, 4 mm and 1.5 mm respectively.

The tensile properties of the dogbone specimens were studied using a universal testing machine (5967 Dual Column testing system, Instron, Norwood, Mass.), installed with a 1 KN capacity load cell. The specimens were fixed in place using serrated-faced metal grips. Specimens were at least stored for 24 hours in ambient conditions before the testing. Testing was conducted at least for 3 samples at ambient conditions at a strain rate of 50 mm/min. Instron's advanced video extensometer (AVE 2663-821) was used to measure the strain (elongation) of the test specimen more accurately by tracking the positions of two contrasting round marks (each near the end of the test regions). Stress-strain analysis was collected for each sample and major tensile parameters, like Young's modulus, percentage elongation at the break, toughness, tensile stress at break and tensile strength were analyzed.

### 2.2.5 IM Tablet Coating

Based upon tensile testing results, particular coating formulations were selected and GF and PEI tablet coating were conducted using the MIT in-house built vertical injection molding machine. This machine had the following components: temperature controlled injection barrel with an orifice in the bottom part, an actuator controlled injection piston, top mold inserts with orifices, bottom mold inserts and a LabVIEW System Design Software (National Instruments, Austin, Tex.). Briefly, the temperature controlled injection barrel, maintained at particular temperature was first filled with the coating formulations. The coating formulation was heated for about 10 minutes to soften it. Two thermocouple heating bands attached at top and bottom part of the barrel maintained the set temperature. The tablets were coated in two steps, in a manner like that described with regard to FIG. 1, and different mold inserts (placed in top and bottom molding halves) were used for each step. Thus, in total 4 mold inserts were used for complete tablet coating. These mold inserts were designed on the basis of the IM tablet shape, size and curvatures, with an aim to provide 300 μm coating thickness. The temperatures of mold inserts were maintained by the circulating liquid cooling system (coolant) in the molding halves. In step 1, the tablet was placed inside the cavity of the mold inserts and the molding halves were closed. Next, injection piston applied pressure to the coating formulation and the applied piston pressure allowed softened coating formulation to progress from the barrel to the mold insert and fill the available space between the tablet and top mold insert. The coating formulation solidified inside the mold cavities in 5-15 seconds and rendered a smooth coating layer attached to the tablet surface. The desired injection pressure, holding pressure, and holding time were controlled with the LabVIEW system design software. Injection pressure was applied by two different modes. The pressure regulated mode targeted to keep the pressure constant by fluctuating the piston position, which resulted in oscillation in pressure values around the target. The position regulated method held the piston in the defined position which allowed the pressure to decay. The typical decrease was about 2000 psi over 5-15 seconds (in Table 2, the higher value is the initial pressure which decayed to the lower value at the end of the cycle). Mold halves were opened and the step 1 coated tablets were collected. The solidified extra coating material (sprue) was removed, the mold inserts were changed, and tablet was flipped over and step 2 coating was performed similar as step 1 to obtain fully coated IM tablet. Step 2 coats overlapped step 1 coats resulting in the coat thickness of 450 μm in the overlapping region.

Process parameters (barrel temperature, injection pressure, mold temperature, and cooling time) were optimized in the following way. Initially, the set point of the barrel temperature was selected to be the same as the miniature twin screw extruder temperature used to prepare the coating formulations. The barrel temperature was further optimized in a way that the complete tablet coating can be achieved at the lowest temperature and injection pressure values. Real time pressure profiles were evaluated each time to ensure the reproducibility of the pressure profiles. Mold temperature and cooling time were then optimized with the aim to minimize the difference between the barrel and mold temperatures. Table 9 discusses the optimized process parameters employed for GF and PEI tablet coating by selected IM coating formulations.

For PVA coat formulation, tablets were coated by a compression molding method. Before closing the mold halves, material was injected to fully cover the top mold cavity of upper mold insert. Mold halves were then closed and coating formulation present on the top mold cavity surface coated the tablet. Then, the mold inserts were changed and tablet was flipped over for step 2 coating. Barrel temperature and mold temperature were maintained at 180° C. and 35° C., respectively. Mold halves were closed for 5-10 sec (hold time).

TABLE 9
IM process parameters employed for GF and PEI tablet coating

| | Coating formulations (polymer + plasticizer w/w) | | | | | | |
|---|---|---|---|---|---|---|---|
| | Hydroxy- | | | | | | |
| | propyl | PEO | PEO | | | | |
| | pea | 100,000 + | 300,000 + | PEO | | | |
| | starch + | 30% | 30% | 1,000,000 + | Opadry + | Opadry + | Opadry + |
| Process | 30% | PEG | PEG | 30% | 20% | 25% | 30% |
| parameters | glycerol | 1500 | 1500 | PEG 1500 | glycerol | glycerol | glycerol |

IM coating - GF tablets

| | | | | | | | |
|---|---|---|---|---|---|---|---|
| Injection pressure | 6000-8000 | 5000-6500 | 5000-7000 | 6000-8000 | 10,000-12,000 | 10,000-12,000 | 10,000-12,000 |
| (psi) | | | | | | | |
| Barrel | 110 | 80 | 95 | 100-130 | 190-200 | 170-180 | 150-170 |
| temperature (° C.) | | | | | | | |
| Mold temperature | 35 | 35 | 35 | 35-55 | 40 | 35 | 35 |
| (° C.) | | | | | | | |
| Hold time (s) | 5 | 5 | 5 | 5 | 15 | 15 | 5-10 |

IM coating - PEI tablets

| | | | | | | | |
|---|---|---|---|---|---|---|---|
| Injection pressure | 6000-8000 | 5000-6500 | 5000-7000 | 6000-8000 | 10,000-12,000 | 10,000-12,000 | 10,000-12,000 |
| (psi) | | | | | | | |
| Barrel | 110 | 80 | 95 | 100 | 180 | 170-180 | 150-170 |
| temperature (° C.) | | | | | | | |
| Mold temperature | 35 | 35 | 35 | 35 | 35 | 35 | 35 |
| (° C.) | | | | | | | |
| Hold time (s) | 5 | 5 | 5 | 5 | 15 | 15 | 15 |

### 2.2.6 Dimensional and Weight Gain Analysis of Coated Tablets

The average weight (with standard deviation) of core and coated tablets were reported for at least five tablets. The core and coated tablet thickness and diameter were measured at least for five tablets using a force-controlled micrometer (Mitoyto, Japan) set to 0.5 N with a resolution of 0.001 mm.

### 2.2.7 Stability Testing

Coated tablets were sealed using an induction sealer (Auto Jr, Enercon industries corporation, Menomonee Falls, Wis.) in high density polyethylene (HDPE 5502BN) pharmaceutical bottles. The bottles were stored in 19° C./<10% RH; 19° C./23% RH; 19° C./33% RH; 19° C./43% RH; 19° C./53% RH; 25° C./45% RH; 25° C./60% RH and 30° C./65% RH storage conditions and evaluated after 4 weeks to evaluate the coat stability. A chamber with dry gas purge was used to create 19° C./<10% RH (typically 2-5% RH) storage condition. Controlled temperature and humidity chambers (LHU-133, Espec, Hudsonville, Mich., USA) were used to provide 25° C./45% RH, 25° C./60% RH and 30° C./65% RH storage conditions. Potassium acetate, magnesium chloride, potassium carbonate and magnesium nitrate saturated salt solutions were used to provide 19° C./23% RH, 19° C./33% RH, 19° C./43% RH and 19° C./53% RH storage conditions.

### 2.2.8 Melt Flow Analysis

A melt flow analysis was conducted using the microinjector with some modifications in the original instrument. The injection barrel was heated to the temperature used to coat the tablets for that particular coating formulation. Extrudates of various coating formulations were cut and manually fed into the heated barrel until it was completely filled. The extrudate pieces were gently pushed down by wooden rod to reduce the voids between them and pack the barrel uniformly each time. The coating formulation was heated for about 10 minutes to soften it. A pressure of 600-630 psi was applied by the injection piston onto the coating formulation residing in the heated barrel and the material coming out from heated barrel was then collected in a container. Typically, the melt flow test is conducted for 10 minutes (indicated as melt flow index) and the result is reported in g/10 minutes unit. However, because of the high melt flow values of hydroxylpropyl pea starch+30% glycerol and limitations in the total barrel volume, the melt flow test was conducted for 1 minute for this particular formulation. For other studied formulations, the test was conducted for 5 minutes and result was converted to g/min units.

### 2.2.9 In Vitro Release Testing

The method for in vitro release testing was based on the USP monograph for GF tablets (Dissolution test 1). USP apparatus II (paddle) dissolution tester (Agilent 708-DS, Agilent Technologies, Santa Clara, Calif.) was employed to evaluate the drug release from uncoated and coated GF tablets. The apparatus vessels were filled with 1000 mL of 40 mg/mL sodium lauryl sulphate. The paddle speed and solution temperature was maintained at 75 rpm and 37±0.5° C. respectively. Samples were collected at specific time intervals, filtered through a 0.45 μm nylon filter, diluted if necessary and evaluated spectro-photometrically by a UV spectrophotometer (Lambda 35, PerkinElmer, Waltham, Mass.) at a wavelength of 291 nm. The average dissolution profile of three tablets were calculated.

### 3. Results and Discussion

### 3.1 Preparation of Coating Formulations and Tensile Testing

The extrudability of each coating polymer, discussed in table 1, was determined without plasticizer. Among these coating polymers, PVA, Kollicoat and Opadry required high processing temperatures (>180° C.) and they sometimes experienced thermal degradation in such stringent processing conditions. Also, very high mechanical energy was required (indicated by higher torque values, sometimes reaching the instrument limit—6.2 Nm), to extrude these polymers. For coating polymer PEO 100,000, the polymer alone could be extruded at low processing temperature (100° C.) but the product was very brittle in nature and it was clear that the polymer would not able to make a robust film layer to coat the tablets. Plasticizer was added to the coating polymers to improve flow and processability and reduce the brittleness of the coating polymer. Plasticizer interposes its molecules between the polymer chains and can also bond with the functional groups of the polymer chains. Thus, it reduces the interaction between the polymer chains and increases the volume between them, imparting chain mobility and flexibility or distensibility. Plasticizers were added in the coating formulations to improve the processability of coating polymers.

Added plasticizer is selected to be compatible and preferably miscible with the polymer, and so most of the selected plasticizers resemble the polymer structure and have the possible interaction capacity with the polymer. Shorter chain polymers (fewer monomers and overall lower M_{w}) can act as plasticizers. Short (M_{w}<100,000) polyethylene oxide polymers, commonly known as PEG's, were selected as plasticizers for PEO. The hydroxyl-containing compound glycerol was used for most coating polymers (having a high hydroxyl ratio) and structurally similar Eudragit NE 30D was employed as a plasticizer for Eudragit E PO. Coating formulations were prepared with particular polymer-plasticizer combinations (table 8) and further screened based upon the physical and visual appearance, uniformity, and scaling issue. Selected formulations (listed in Table 10) were analyzed for tensile properties. Table 10 discusses the calculated values of tensile properties, like Young's modulus, percentage elongation, toughness, tensile stress at break and tensile strength (also called ultimate strength or ultimate tensile strength).

### 3.1.1 Young's Modulus

Young's modulus, also called the elastic modulus, is estimated from the slope of the linear region of the stress-strain profile where the formulation experiences elastic deformation. This fundamental material property shows the elasticity of the film with lower values corresponding to higher elasticity. It evaluates the specimen resistance to the elastic deformation. The values are directly related to the interatomic bonding energy, with higher values corresponding to the stiffer and rigid film where it needs higher loads to deform elastically. Prima facie, it was hypothesized that the coating polymers having low values of Young's modulus should be good for IM coating. The employed methodology differentiated between the coating formulations, considering their capability to resist the deformation. The coating formulations containing PVA, Kollicoat, hydroxypropyl pea starch and Opadry had significantly lower Young's modulus values (Table 10) compared to the other coating polymers employed in the study. As plasticizer is expected to reduce the stiffness of the polymer, increase in plasticizer content decreased the Young's modulus values of coating polymers PVA and Opadry. However, there was no particular trend for PEO when plasticizer (PEG) amount increased from 10% to 30%.

### 3.1.2 Elongation (%)

Coating formulations containing high molecular weight PEO (300,000 and 1,000,000), PVA, Opadry and hydroxypropyl pea starch had significant elongation in comparison with Eudragit E PO, Kollicoat, and PEO (100,000). The stretching or elongation is expected to be increased with an increase in molecular weight of the polymer and the plasticizer amount employed for the same polymer. There was an increase in percentage elongation with an increase in molecular weight of PEO. For Opadry and PEO (300,000), increasing the plasticizer amount further increased the percentage of elongation. However, for PVA, an increase in plasticizer amount did not change percentage elongation. For the studied coating formulations, PVA, Opadry, PEO and hydroxypropyl pea starch based formulations showed higher percentage elongation values (table 10) when high plasticizer content was employed.

### 3.1.3 Tensile Strength

Tensile strength is the maximum force per unit area applied to the specimen. It is the maximum stress that a specimen can withstand before necking or cracking. Plasticizers weaken the intermolecular forces between the polymer chains, which typically reduces the tensile strength and brittleness. In case of PVA and Opadry, tensile strength was reduced with the addition of plasticizer as per the expectations. However, it was not the case for PEO-PEG system.

### 3.1.4 Toughness

Toughness, the total area under the stress-strain curve, is a measurement of the energy absorption before failing. Toughness indicates material's resistance to breakage. Toughness of the specimen depends on both the strength and ductility of the specimen. Based upon the results obtained, PVA, Opadry, PEO and hydroxypropyl pea starch based formulation showed higher toughness values (because of higher strength or ductility or the combination of both).

### 3.1.5 Tensile Stress at Break

The stress at the moment when the test specimen breaks is considered as the tensile stress at break. This mechanical parameter was also determined for all coating formulations.

TABLE 10A
Tensile properties of IM coating formulations

| | | Tensile properties of coating formulations, | | |
|---|---|---|---|---|
| | Microinjector | measured at ambient conditions | | |
| | barrel | Tensile | | Ratio - Tensile |
| | temperature | stress at | Tensile strength | strength/Young's |
| Coating formulation | (° C.) | break (MPa) | (MPa) | modulus |
| PEO 100,000 + 30% PEG 1,500 | 80 | 9.09 (0.41) | 15.73 (1.04) | 0.013 |
| PEO 300,000 + 10% PEG 1,500 | 90-110 | 2.20 (1.33) | 10.1 (2.0) | 0.018 |
| PEO 300,000 + 20% PEG 1,500 | 90-110 | 2.97 (2.74) | 11.7 (2.0) | 0.022 |
| PEO 300,000 + 30% PEG 1,500 | 90-110 | 6.43 (1.23) | 11.5 (1.0) | 0.020 |
| PEO 1,000,000 + 30% PEG 1,500 | 90-110 | 1.97 (0.49) | 11.29 (0.01) | 0.017 |
| PVA + 20% Glycerol | 190 | 22.36 (3.27) | 42.4 (1.2) | 0.372 |
| PVA + 30% Glycerol | 170 | 11.60 (1.05) | 21.3 (1.5) | 0.260 |
| PVA + 40% Glycerol | 150 | 11.00 (1.21) | 11.6 (1.0) | 0.240 |
| Eudragit E PO + 40% Eudragit | 125 | 12.89 (0.71) | 23.1 (1.7) | 0.010 |
| NE 30D | | | | |
| Kollicoat + 20% glycerol | >150 | 2.47 (0.86) | 5.0 (1.0) | 0.032 |
| Hydroxypropyl pea starch + 30% | 100-130 | 0.07 (0.10) | 0.49 (0.12) | 0.312 |
| Glycerol | | | | |
| Opadry + 20% Glycerol | 190 | 8.16 (2.16) | 16.6 (3.5) | 0.053 |
| Opadry + 25% Glycerol | 170 | 5.47 (0.17) | 9.53 (0.31) | 0.099 |
| Opadry + 30% Glycerol | 150 | 3.41 (0.22) | 5.96 (0.45) | 0.089 |

| | | |
|---|---|---|
| PVA + PEG 400* | ~170 | Chalky white product, incompatible plasticizer |
| PVA + PEG 1500* | ~170 | Chalky white product, incompatible plasticizer |
| Kollicoat + PEG 400* | >150 | Poor melt flow, product breaks apart, |
| | | incompatible plasticizer |
| Kollicoat + PEG 1500* | >150 | Poor melt flow, sticks in mold cavity, |
| | | incompatible plasticizer |

*10, 20 and 30% of PEG were used, values in parenthesis indicate standard deviations

TABLE 10B
Tensile properties of IM coating formulations

| | | Tensile properties of coating | | |
|---|---|---|---|---|
| | | formulations, measured at ambient | | |
| | Microinjector | conditions | | |
| | barrel | Young's | | |
| | temperature | Modulus | Elongation | Toughness |
| Coating formulation | (° C.) | (MPa) | (%) | (J/m3 * 10⁴) |
| PEO 100,000 + 30% PEG 1,500 | 80 | 1209 (141) | 2.14 (0.81) | 22.9 (7.1) |
| PEO 300,000 + 10% PEG 1,500 | 90-110 | 571 (39) | 11.0 (2.9) | 86.6 (17.3) |
| PEO 300,000 + 20% PEG 1,500 | 90-110 | 517 (22) | 15.2 (6.5) | 126 (67) |
| PEO 300,000 + 30% PEG 1,500 | 90-110 | 572 (41) | 15.6 (6.2) | 187 (73) |
| PEO 1,000,000 + 30% PEG 1,500 | 90-110 | 656 (65) | 51.6 (7.9) | 370 (72) |
| PVA + 20% Glycerol | 190 | 114 (22) | 90.9 (14.6) | 2724 (903) |
| PVA + 30% Glycerol | 170 | 81.8 (4.9) | 88.5 (3.6) | 1440 (74) |
| PVA + 40% Glycerol | 150 | 49.3 (4.4) | 88.4 (14.2) | 834 (200) |
| Eudragit E PO + 40% Eudragit | 125 | 2200 (302) | 1.37 (0.13) | 21.3 (4.2) |
| NE 30D | | | | |
| Kollicoat + 20% glycerol | >150 | 157 (12) | 5.31 (1.11) | 16.0 (1.5) |
| Hydroxypropyl pea starch + 30% | 100-130 | 1.57 (0.20) | 230 (19) | 97.1 (26.4) |
| Glycerol | | | | |
| Opadry + 20% Glycerol | 190 | 309 (56) | 16.9 (2.9) | 181.6 (30) |
| Opadry + 25% Glycerol | 170 | 95.8 (5.2) | 35.80 (0.85) | 218 (20) |
| Opadry + 30% Glycerol | 150 | 66.6 (5.2) | 30.00 (0.42) | 144 (10) |

*10, 20 and 30% of PEG were used, values in parenthesis indicate standard deviations

### 3.2 IM Tablet Coating

Based upon tensile testing analysis and values of the microinjector barrel temperature, the coating polymers in Table 2 were selected for IM coating. Eudragit E PO and Kollicoat based formulations had very low percentage elongation values. Also, Eudragit E PO based formulation was rigid and the Kollicoat based formulations required higher process temperatures and had severe sticking to mold surface; therefore, they were not used further. For PEO 300,000 based formulations, PEO 300,000+30% PEG 1500 was chosen since it had improved mechanical properties compare to the PEO based formulation employing 10% or 20% PEG 1500. Addition of plasticizer increases the energy necessary to initiate the crack. Plasticizer decreases Young's modulus and glass transition temperature of the specimen, effectively reducing the internal stress and decreasing the incidence of cracking. PVA+20% glycerol required >190° C. processing temperature whereas PVA+40% glycerol extrudates and specimens, stored in ambient conditions showed phase separation of glycerol from PVA in a week and therefore PVA+30% glycerol was selected for coating.

IM is a very complex process which requires a sound understanding of IM process parameters and material attributes. Process optimization to ensure the final molded product robustness requires optimization of IM input variables such as barrel temperature, mold temperature, injection pressure and cooling time.

The barrel temperature is one of the most critical parameters to ensure proper flow of the material and therefore the IM product quality. Another critical parameter is the injection pressure employed to inject the coating formulation from the barrel to the mold cavity. A pressure regulated mode was employed first. In order to keep the pressure constant (as discussed in section 2.2.5), the fluctuations in pressure around the desired value caused deformation in the tablet cores. Therefore, a position regulated mode was developed to coat the tablets. In this mode, the decay in pressure during injecting/holding/cooling mode of the coating material in the mold cavity prevented any deformation to the core. This position regulated method was hence selected for carrying out the coating experiments. Low injection pressure could result in incomplete mold filling, whereas, high injection pressure could generate pressure induced stress and flashing. Barrel temperature and injection pressure were therefore optimized with the utmost priority. Initially, the set point of the barrel temperature was the same as the miniature twin screw extruder temperature used to formulate the coating material. The barrel temperature was further optimized to achieve a complete tablet coat at the lowest temperature and injection pressure. Real time pressure profiles were evaluated each time to ensure the reproducibility of pressure profiles. Mold temperature and cooling time were then optimized with the aim to minimize the difference between the barrel and mold temperatures while not being excessively long. The quality of the final product is greatly affected by the cooling stage of the injection molding cycle wherein a hot melted polymer is injected into the mold and allowed to stay until it solidifies. Optimization of cooling time and the mold temperature plays an integral role for good coating. During the cooling stage, heat transfer also affects crystallization kinetics, shrinkage, and residual stresses and thereby impacts the mechanical properties, surface clarity and geometric tolerance. Therefore, considerable importance was given to these process parameters as well. Table 2 provides the optimized process parameters employed for IM coating. For the selected PVA formulation, high pressure and temperature were not sufficient enough to coat the tablet by IM. PVA based coating formulation was not flowing properly even at high temperature and pressure. Therefore, compression molding was used to coat the tablets for this formulation. The melt flow was analyzed for 4 coating formulations (discussed herein).

Hydroxypropyl pea starch, Opadry, PEO based formulations provided uniform coating for both types of tablets, whereas non-uniform thick coating were obtained with PVA based coating formulation (due to the compression molding process). Room moisture was critical while coating tablets with PEO based formulations, particularly with PEO 1000,000+30% PEG. In dry condition (19° C., <30% RH), PEO 1000,000+30% PEG coated tablets cracked within an hour of coating, for both types of tablets. When room humidity was higher than 30% RH, stable coats were obtained for PEI tablets. However, immediate coat cracking was sometime observed for GF tablets and it could be due to the pressure induced stress.

In preliminary stability studies, tablets in sealed bottles were stored in ambient conditions (19° C., 40-60% RH), 19° C./<10% RH, 25° C./45% RH and 30° C./65% RH. Within 2 days, PEO 100,000 and 300,000 based coated formulations cracked in all storage conditions. GF and PEI core tablets did not experience any dimensional instability and it should not be the reason for these cracking. These 2 coating formulations had poor tensile properties compare to PEO 1000,000. Also, an increase in the molecular weight of the polymer provides tougher coating with a decrease in the incidences of cracking as well as the crack propagation. This could be the reason for the cracking of coats made from PEO 100,000 and 300,000 based formulations. In all the storage conditions, Opadry+30% glycerol formulation experienced phase separation within 7 days with glycerol leaching out from the Opadry. Although Opadry+20% glycerol formulation was stable in all storage conditions, the coat was not so smooth. Opadry+25% glyceol was found to be stable as well as produced smooth coats. Tablets coated with PEO 1000,000, hydroxypropyl pea starch and Opadry had stable coating without cracks (7 days, 3 storage conditions). The coating weight gain and dimensions of the tablets are provided in Table 11. The coating weight gain was higher for GF tablets in comparison with PEI tablets. This slight difference in weight gain could be due to the dimensional differences between PEI and GF tablets. It was also observed that the maltodextrin based GF tablets were bonded strongly with coating formulations. As per the mold design, the increase in tablet thickness was ˜0.6 mm (2*0.3 mm coating on both side of the tablet) and the increase in tablet diameter was ˜0.9 mm (2*0.45 mm). Based upon these preliminary studies, tablets (PEI and GF) coated with hydroxypropyl pea starch and Opadry as well as tablets (PEI) coated with PEO 1000,000 based formulations were stored in various stability conditions (listed in section 2.2.7) and further evaluated.

TABLE 11
Weight, thickness (t) and diameter (d) of coated tablets

| | | Coated tablet features | | | | | |
|---|---|---|---|---|---|---|---|
| | | | Weight | | | | Increase |
| | Coating | Weight | gain | | Increase | | in d |
| Tablet | formulation | (mg) | (mg) | t (mm) | in t (mm) | d (mm) | (mm) |
| PEI (weight | Opadry 200 + | 597 ± 1 | 137 | 6.263 ± 0.019 | 0.563 | 10.928 ± 0.010 | 0.928 |
| 460 ± 5 mg, | 25% Glycerol | | | | | | |
| thickness 5.70 mm, | Hydroxypropyl | 586 ± 1 | 126 | 6.264 ± 0.017 | 0.564 | 10.930 ± 0.014 | 0.930 |
| diameter | pea starch + | | | | | | |
| 10.0 mm) | 30% Glycerol | | | | | | |
| | PEO | 570 ± 2 | 116 | 6.265 ± 0.013 | 0.565 | 10.997 ± 0.018 | 0.997 |
| | 1,000,000 + 30% | | | | | | |
| | PEG 1,500 | | | | | | |
| GF* (weight | Opadry 200 + | 679 ± 1 | 159 | 6.311 ± 0.019 | 0.611 | 10.930 ± 0.014 | 0.930 |
| 523 ± 1 mg, | 25% Glycerol | | | | | | |
| thickness 5.67 mm, | Hydroxypropyl | 666 ± 2 | 143 | 6.322 ± 0.008 | 0.623 | 10.896 ± 0.019 | 0.896 |
| diameter | pea starch + | | | | | | |
| 10.0 mm) | 30% Glycerol | | | | | | |

*data for PEO 1,000,000 + 30% PEG 1,500 formulation is not provided as some tablet coating immediately cracked after the coating process.

### 3.3 Stability Testing

Hydroxypropyl pea starch coated GF and PEI tablets did not show cracking for all storage conditions for 4 weeks. Opadry coated PEI tablets were also stable in all storage conditions. Opadry based GF tablets were stable at and below 45% RH (19° C. and 25° C.). However, these tablets showed phase separation of glycerol from Opadry when stored above 45% RH. Apart from the storage temperature and humidity, GF core tablets had critical role in this separation as the same phase separation was not observed for the PEI tablets. GF cores have moisture sensitive maltodextrin as polymer matrix and based upon our loss on drying experiments, these tablets have ˜0.5% residual moisture content. Aggressive storage conditions and the tablet residual moisture induced this phase separation. PEO 1,000,000 based coats were stable with PEI tablets when stored at 19° C./43% RH; 19° C./53% RH; 25° C./45% RH; 25° C./60% RH and 30° C./65% RH but cracked at lower humidity levels. Optimized processing and storage stability conditions suggest that PEO based formulations need a certain amount of moisture in polymer coats to prevent cracking.

### 3.4 Melt Flow Analysis

TABLE 12
Melt flow analysis of coating formulations

| | Melt | Temperature |
|---|---|---|
| Coating formulation | flow (g/min) | (° C.) |
| Hydroxypropyl pea starch + 30% Glycerol | 5.35 ± 0.14 | 110 |
| Opadry 200 + 25% Glycerol | 0.56 ± 0.09 | 170 |
| PEO 1,000,000 + 30% PEG 1,500 | 0.44 ± 0.05 | 130 |
| Polyvinyl alcohol + Glycerol (30%) | 0.11 ± 0.02 | 165 |

Melt flow analysis is an important quality control rheological parameter in the plastic industry which gives the critical data and interpretation about the suitability and processability of thermoplastic polymers for IM.

Coating formulations tested for their melt flow and the resultant melt flow values are tabulated (Table 12). Hydroxylpropyl pea starch+30% glycerol showed the highest melt flow followed by Opadry 200+25% glycerol and PEO 1,000,000+30% PEG 1500. PVA+30% glycerol had significantly low melt flow values, suggesting the poor processability and confirming the reason of difficulties experienced during IM processing (despite exploring all the IM processing conditions). Thus, the melt flow test helped to predict, understand, and correlate processability of coating formulations for IM tablet coating application.

3.5 Discussion about Coat Stability and its Relationship with Tensile Testing, Melt Flow Analysis, and Core Formulations

Overall, the following 8 major observations were found from the coating experiments and stability studies and they can be correlated with the tensile testing and melt flow analysis.

First, coating formulations (for example, Opadry based) with good melt flow characteristics could coat the temperature and pressure sensitive maltodextrin based GF tablets even though the processing temperature was very high (170-180° C.). Good melt flow of the coating formulation compensated for the high processing temperature and the tablet was able to endure the effect of temperature, pressure and did not deform.

Second, injection pressure should be controlled such that the pressure does not oscillate during the injection cycle. Oscillation caused the deformation of tablet cores whereas, unfluctuating pressure pattern eliminated core deformation when optimized pressure and temperature profiles were employed.

Third, typically, the formulations with lower Young's modulus (for example, hydroxypropyl pea starch and Opadry 200 based) provided suitable IM coating with stable coats. A low Young's modulus is advantageous in averting initiation and propagation of cracks.

Fourth, coating formulations with higher percentage elongation (>35%) values were found to be more robust for IM coating.

Fifth, toughness was found to be a good indicator to look for in screening formulations for IM coating. Overall, formulations having toughness values higher than 95×10⁴ J/m³ (for example, Opadry, PVA, hydroxypropyl pea starch and PEO 1,000,000 based formulations) performed well in IM coating. The major exception was PEO 300,000+30% PEG 1,500, which cracked after storage and lower ductility (elongation) could be the reason.

Sixth, tensile stress at break is not an acceptable indicator for IM coating formulation selection as there was no clear correlation between the tensile stress at break values and IM processing as well as coat stability.

Seventh, the derived mechanical parameter, tensile strength/Young's modulus ratio indicates crack resistance and could predict cracking. Based upon the requirement of the tough and elastic nature of the tablet coat, coating formulations having a high ratio of tensile strength/Young's modulus would resist the external forces and stresses and have a lower tendency towards the cracking. PVA, hydroxypropyl pea starch, and Opadry based formulations had high values compared to other coating formulations and these coating formulations were the most successful in IM coating.

Eighth, maltodextrin based GF tablets could be deformed at high injection pressure and would be sensitive to processing temperature, humidity, and pressure induced residual stress mainly because of the sensitive polymer matrix (maltodextrin) employed to formulate the IM tablets. To eliminate these confounding factors, temperature and pressure resistant, as well as moisture insensitive PEI tablets were employed in the study. PEO based coating formulation provided an acceptable and stable IM coating for PEI tablets and confirmed the feasibility of this formulation for IM coating. However, the coatings cracked when applied to maltodextrin based GF tablet, corroborating the fact that the core tablets play a role in successful IM coating.

For successful pharmaceutical tablet coating, a formulator can work on the basis of two approaches, minimize the internal stress of the system or accept these internal stresses and minimize the incidence of the defect by formulating “right” coating formulation that can absorb these stresses and survive. It seems we moreover applied the combined approach where we first selected the “right” coating formulations with the help of tensile testing and later prevented the coating defects (mainly cracking) by optimizing the IM processing. For researchers working with IM coating, it is recommended that the tensile testing and melt flow analysis would be initially helpful to screen the coating formulation, followed by IM processing parameter optimization.

### 3.6 In Vitro Release Study

In vitro dissolution study was conducted for tablets coated with hydroxypropyl peastarch+30% glycerol, Opadry+25% glycerol and PEO 1000,000+30% PEG 1500 formulations. Greater than 75% drug was released from uncoated GF tablets in less than 15 minutes. Next, tablets coated with hydroxypropyl peastarch+30% glycerol had a good immediate release profile with >75% drug was released in ˜25 minutes. Hydroxypropyl pea starch is a water-soluble polymer and an addition of glycerol as a plasticizer to hydroxyporpyl peastarch coating could help to further increase the water solubility. It has been reported in the literature that, as the concentration of glycerol in the peastarch formulations increase, more OH groups are available for hydrogen bonding and it increases the solubility of peastarch.

PEO 1000,000+30% PEG 1500 also provided an immediate release profile for GF tablets (>75% drug release in ˜30 minutes). This could be attributed to good solubility of PEO in water and thereby helping the dissolution. It also has a capacity to swell and erode when placed in the dissolution media.

Opadry+25% glycerol required 50 minutes to dissolve >75% of GF. As per USP monograph of griseofulvin tablets (test 1), >75% drug should be dissolved in less than 90 minutes. Thus, it still complies the USP monograph. However, the release was slower in comparison with hydroxypropyl pea starch and PEO based formulations. Chemically, Opadry is a PVA based polymer and the solubility profile of PVA depends upon the degree of hydrolysis and molecular weight. Since the label of Opadry only mentions it as a PVA based polymer and details could not be found about its hydrolyzation or molecular weight, the reason for this poor dissolution profile is difficult to justify. Also, the thick coat (300 μm thickness), obtained by IM coating, slowed down the drug release. A decrease in coating thickness would improve drug release of tablets coated by all coating formulations.

### Supplementary Humidity Analysis

Coated tablets were stored in open containers at 19° C./<10% RH; 19° C./23% RH; 19° C./33% RH; 19° C./43% RH; 19° C./53% RH; 25° C./45% RH; 25° C./60% RH and 30° C./65% RH storage conditions and evaluated after 4 weeks to evaluate the coat stability. GF and PEI tablets coated with hydroxypropyl pea starch did not show any cracks stored in all temperature and RH conditions for 4 weeks. PEO 1,000,000 coated PEI tablets were stable for 4 weeks in 19° C./43% RH; 19° C./53% RH; 25° C./45% RH; 25° C./60% RH and 30° C./65% RH; but cracked in lower humidity conditions as the coating formulation required a certain moisture amount to prevent cracking. PEI tablets coated with Opadry 200 based formulations were stable in 19° C./10% RH, 19° C./23% RH, and 19° C./33% RH storage conditions. Whereas, glycerol experienced phase separation from Opadry when these tablets were stored at 19° C./43% RH, 19° C./53% RH, 25° C./45% RH, and 25° C./60% RH (higher moisture conditions). Direct exposure to moisture most likely softened the material, allowing for higher mobility and accelerated the phase separation in tablets stored in open containers compared to the sealed containers.

### 4. Conclusion

Material properties (Young's modulus, toughness, percentage elongation, and tensile strength/Young's modulus ratio), obtained from the stress-strain analysis helped in screening the coating formulations suitable for IM process. The melt flow characteristics of the coating formulations played a vital role in IM processing. Injection pressure, barrel temperature and mold temperature were identified as critical process parameters for IM coating and were evaluated in detail. Based upon this study, hydroxypropyl peastarch+30% glycerol, Opadry+25% glycerol and PEO 1,000,000+30% PEG were concluded as viable coating formulations for IM based tablet coating. These formulations possessed the mechanical and material properties required by IM processing, rendered stable coats and desired dissolution profile. The study proved that IM is a promising technology for tablet coating. The study also serves as a model for product development with specifications of excipients in ranges within the designed acceptance space for optimal product performance.

### Example 5

This Example provides a framework for robust tablet development using an integrated hot-melt extrusion-injection molding (IM) continuous manufacturing platform. Griseofulvin, maltodextrin, xylitol and lactose were employed as drug, carrier, plasticizer and reinforcing agent respectively. A pre-blended drug-excipient mixture was fed from a loss-in-weight feeder to a twin-screw extruder. The extrudate was subsequently injected directly into the integrated IM unit and molded into tablets. Tablets were stored in different storage conditions up to 20 weeks to monitor physical stability and were evaluated by polarized light microscopy, DSC, SEM, XRD and dissolution analysis. Optimized injection pressure provided robust tablet formulations. Tablets manufactured at low and high injection pressures exhibited the flaws of sink marks and flashing respectively. Higher solidification temperature during IM process reduced the thermal induced residual stress and prevented chipping and cracking issues. Polarized light microscopy revealed a homogeneous dispersion of crystalline griseofulvin in an amorphous matrix. DSC underpinned the effect of high tablet residual moisture on maltodextrin-xylitol phase separation that resulted in dimensional instability. Tablets with low residual moisture demonstrated long-term dimensional stability. This study serves as a model for IM tablet formulations for mechanistic understanding of critical process parameters and formulation attributes required for optimal product performance.

### Introduction

The pharmaceutical community has realized the need for new manufacturing technologies and is advancing towards the next phase of modernization by shifting from batch to continuous manufacturing. Minimization of scale up requirements, reduced space, energy and carbon foot-print, reduced processing time, minimized manufacturing cost and an increase in process efficiency, and product quality are benefits of this paradigm. Regulatory agencies, such as United States Food and Drug Administration (US-FDA) and European Medicines Agency (EMA), have also echoed this initiative firmly. Pharmaceutical industries, regulatory agencies, academicians and researchers have reached the consensus that the continuous manufacturing can often have a significant edge over batch manufacturing. Traditional batch methods of drug product conversion often involve multiple costly and time consuming powder handling steps such as milling, wet or dry granulation, drying, sieving, and tableting to produce a uniform product. Continuous drug product manufacturing decreases process and handling steps via innovative integration of excipients and APIs. Toward this goal, the Novartis-MIT Center for Continuous Manufacturing has developed an integrated hot-melt extrusion (HME) and injection molding (IM) process.

HME is a continuous melt processing technology that is widely used in the plastic industry and involves the mixing of polymers, carriers and other constituents with the application of heat and shear. It is a solvent-free technique that can be utilized in the pharmaceutical industry to produce homogeneous mixtures of APIs and excipients under elevated temperatures and shear. HME Process parameters (particularly barrel temperature, screw design, screw speed and feed rate) in addition to the native formulation attributes influence API melting and dispersion in a polymer matrix. HME processing can often increase the solubility and bioavailability of poorly soluble APIs such as by transforming the API to an amorphous state. However, it is understood that the amorphous form is often thermodynamically unstable which can spontaneously transform to a more stable crystalline form upon storage or in vivo after ingestion. Crystalline solid dispersion, on the other hand, would be more stable and a well dispersed crystalline API in water soluble or hydrophilic polymer matrix (in other words, microfine crystalline dispersions) could still improve the dissolution of API.

IM is a rapid, melt processing-based and versatile technology to manufacture products of diverse and intricate three dimensional shapes with high precision. The quality of an IM product relies on different factors such as part design, mold design, material attributes and process parameters. Process parameters such as injection pressure, hold pressure, mold surface temperature, and cooling time are critical in achieving a robust IM product. This complicated multi-physical process imparts thermal and mechanical history including molding defects, like dimensional deviations, flashing, short shot, etc. However, the HME-IM platform is relatively new and critical process parameters (CPPs) and formulation attributes affecting IM products have not been thoroughly explored. A systematic investigation of these process parameters and formulation attributes could prevent common tableting defects associated with IM tableting. Tablets manufactured from the optimized formulation described above also experienced dimensional instability and as per our best knowledge, the root causes behind this issue have not been studied. These deviations could be detrimental to subsequent downstream pharmaceutical processing steps such as coating, where coating defects could appear due to the core tablet expansion. Considering the HME process perspective for pharmaceutical applications, the physical stability of solid dispersions has always been an associated concern. Physical instability often appears due to phase separation between formulation constituents into distinct phases and it would be imperative to study how this plausible phase separation phenomenon affects injection molded tablets. Furthermore, studies of storage conditions and subsequent tablet stability (manufactured by the integrated HME-IM platform) have been scarce. Additionally, HME and IM operations were accomplished separately in most reported cases. To achieve a successful integrated HME-IM continuous manufacturing platform, these shortfalls must be addressed.

In this examination, an integrated HME-IM platform was used to continuously manufacture tablets. Griseofulvin, maltodextrin, xylitol and lactose were employed as model drug, polymer matrix, plasticizer and reinforcing agent, respectively. CPPs and key performance metrics of this recently introduced platform were identified and rigorously evaluated to achieve robust tablet manufacturing with acceptable product properties and performance. Herein, dimensional changes of the injection molded tablets were thoroughly analyzed and root causes responsible for these changes were underpinned in detail. This study employed a wide combination of microscopic, thermal and spectroscopic characterization tools to evaluate phase transition and separation phenomena. Lastly, we investigated the effect of environmental conditions on the overall stability of the formulated product.

### Materials and Methods

### 2.1 Materials

Griseofulvin (USP) was purchased from Jinlan Pharm-Drugs Technology Co. Limited. (Hangzhou, China). Maltodextrin (Glucidex IT 12) and xylitol (Xylisorb® 90) were donated by Roquette America Inc. (Geneva, Ill., USA). Anhydrous lactose (SuperTab 24AN) was obtained from DFE Pharma (Paramus, N.J., USA). Potassium acetate and magnesium nitrate hexahydrate were purchased from Sigma-Aldrich, Co. (St. Louis, Mo., USA).

### 2.2 Methods

### 2.2.1 IM Tablet Manufacturing by HME-IM Processing

Integrated HME-IM was performed with a formulation based on (a physical mixture of) griseofulvin (10% w/w), maltodextrin (54.4% w/w), xylitol (32.6% w/w) and lactose (3% w/w). The components (batch size, 400 g) were screened through an 800 μm sieve and mixed using a shaker mixer (Turbula® T2F, Glen Mills Inc, Clifton, N.J., USA) for 10 minutes. Then, the mixture was fed through a gravimetric (loss-in-weight) feeder at 80 g/h into an intermeshing co-rotating 16-mm twin screw extruder (Nano 16, Leistritz, Somerville, N.J., USA). The screw speed was set to 90 rpm while the inlet zone temperature was set to 8° C., to prevent premature melting of the mixture. Zones one, two, three and four of the barrel were heated to 80° C., 155° C., 155° C. and 155° C., respectively. The screw design consisted of different segmented screw elements (120 mm wide conveying elements, 150 mm narrow flight conveying elements, 30 mm kneading block, 60 mm wide flight conveying elements and 60 mm narrow flight conveying elements) along the length of the screw. This screw configuration provided sufficient mechanical shear to the mixture. Dimensions of the screws, given in terms of length (L) to diameter (D) ratio, were 25:1. The melt temperature, melt pressure and torque values were monitored in real time throughout the run. The outlet of the extruder was coupled to an IM unit (MHS Hot Runner Solutions, Ontario, Canada) via a 0.6 cm cylindrical exit die. The IM unit consists of two main temperature controlled regions: a reservoir and a hot-runner section. The hot-runner zone can be further divided into manifold, injection nozzle, and valve gate area. The molten extrudate mass were directly flushed through this cylindrical exit die into the heated reservoir. The mass would further travel from the reservoir to manifold, injection nozzles and six valve gates and shape into the tablets inside the six mold cavities. The reservoir, manifold and injection nozzle were set at 150° C., 145° C. and 130-135° C. respectively.

IM is a repeated processing and can be divided into four phases: filling phase, packing phase, holding phase, and cooling phase. In the filling phase, material is injected into the mold cavity at a particular injection pressure. In the packing phase, material will continue to flow into the cavity to fill any voids which form due to material shrinkage resulting from the transformation of a melt to a solid. Next is the holding phase where the injected material, present inside the cavity, will be held at particular pressure and time. Finally, in the cooling phase, the molten material sufficiently solidifies so that the final product can be ejected from the cavity. In this study, injection pressure and mold surface temperature were the critical parameters and therefore were studied in detail. Both parameters were varied at different levels and the resultant IM product was evaluated. After preliminary studies, it was found that the injection time, hold pressure and hold time did not affect IM processing and product quality and were therefore kept constant. The reservoir back pressure and cooling time were adjusted in a particular range, to control the reservoir filling and solidification of the tablets. Table 13 summarizes the process parameters and their values used throughout the study.

TABLE 13
IM process parameters and their values employed in integrated
HME-IM platform

| IM Process parameters | Values or range |
|---|---|
| Injection pressure (psi) | 1300, 1630, 1960, 2285, 2610, 2940 |
| Back pressure (psi) | 360-470 |
| Injection time (s) | 1 |
| Hold pressure (psi) | 1140 |
| Hold time (s) | 0.5 |
| Mold surface temperature (° C.) | 30, 35, 40, 45 |
| Cooling time (s) | 15-30 |

In a nutshell, a single parameter method was used where only injection pressure or mold surface temperature were varied one parameter at a time, keeping other parameters constant. This provided valuable guidance about high quality product manufacturing with the lowest rejection ratio. Injection molded tablets were stored in open containers at 19° C./<10% RH, 25° C./45% RH and 30° C./65% RH and in high density polyethylene (HDPE 5502BN) pharmaceutical bottles at 19° C./<10% RH, 19° C./23% RH, 19° C./53% RH, 25° C./45% RH, 30° C./65% RH and 40° C./75% RH to assess their physical stability (appearance, tablet dimensions, crystallinity, thermal analysis, and water uptake). A nearly sealed chamber with a dry gas purge was used to create a 19° C./<10% RH storage condition. Controlled temperature and humidity chambers (LHU-133, Espec, Hudsonville, Mich., USA) were used to provide 25° C./45% RH, 30° C./65% RH and 40° C./75% RH storage conditions. Potassium acetate and magnesium nitrate saturated salt solutions were used to provide 19° C./23% RH and 19° C./53% RH storage conditions.

### 2.2.2 Characterization of Formulation Constituents and Injection Molded Tablets

### 2.2.2.1 X-Ray Diffraction (XRD)

A Panalytical MPD X'Pert Pro (Bruker, Madison, Wis., USA) with copper K-alpha radiation (1.541 Å) at 45 kV and 40 mA was employed to obtain XRD patterns periodically at ambient temperature. Powder constituents and fragmented tablets (22±2 mg) were scanned over a range of 20 values from 3 to 40°, with a step size of 0.008° and step time of 25 sec. HighScore Plus diffraction software (Panalytical) was used to further analyze the obtained diffractograms.

### 2.2.2.2 Differential Scanning Calorimetry (DSC)

Thermal behavior of IM tablets was analyzed using a DSC (DSC Q2000, TA Instruments, New Castle, Del., USA) calibrated with indium. Samples (4-6 mg) were placed in Tzero aluminum crimped pans with pin-holed hermetic lids. Thermograms for powder constituents were obtained using the conventional DSC (cDSC) technique, where samples were equilibrated at −20° C. and heated from −20° C. to 260° C. at 10° C./min. Additionally, fragmented tablets were equilibrated at −20° C., and a modulated DSC (mDSC) method was performed periodically from −20° C. to 260° C. at 2° C./min heating rate with a 0.5° C. modulation amplitude for 60 seconds. For all experiments, nitrogen was purged at 50 mL/min. Thermographs were analyzed using the Universal Analysis 2000 Software (TA Instruments). The drug melting endotherm, enthalpy relaxation, derivative of reversible heat flow signal and the area under the curve (AUC) of the derivative reversible heat flow signal were evaluated.

### 2.2.2.3 Polarized Light Microscopy

Tablet sections were obtained using a microtome (Leica, EM UC6, Buffalo Grove, Ill., USA). The sections were placed on the glass slide, covered by coverslip and then examined for birefringence using an optical microscope (Olympus BX51M, PA, USA) fitted with a polarizer, with an objective of 20× and an ocular magnification of 10×. Observations were captured using the camera mounted on the microscope. Images captured details of both crystalline and amorphous domains.

### 2.2.2.4 Weight and Dimension Analysis

The average weight (with standard deviation) was reported for twenty tablets. The tablet thickness and diameter were measured using a force-controllable micrometer (Mitutoyo, Kawasaki, Japan) set to 0.5 N with a resolution of 0.001 mm. The average thickness and diameter were reported at least for five tablets.

### 2.2.2.5 In Vitro Release Studies

In vitro release testing was based on the USP monograph for griseofulvin tablets (Dissolution test 1). Drug release was evaluated using a USP apparatus II (paddle) dissolution tester (Agilent 708-DS, Agilent Technologies, Santa Clara, Calif., USA) filled with 1000 mL of 40 mg/mL sodium lauryl sulphate. The paddle speed was 75 rpm and the solution temperature was maintained at 37±0.5° C. Samples were withdrawn at particular time intervals, filtered through a 0.45 μm nylon filter, diluted if required and spectro-photometrically assessed by means of a UV spectrophotometer (Lambda 35, PerkinElmer, Waltham, Mass., USA) at a wavelength of 291 nm. The average dissolution profile of three tablets were reported.

### 2.2.2.6 Loss on Drying (LOD)

Residual moisture content was evaluated using a Sartorius Moisture Analyzer (MA 100, Sartorius GmbH, Germany). IM tablets were crushed and a known amount of crushed particle mass was heated in an isolated chamber on a balance that measured the weight loss (representing moisture content) at 160° C. for 45 minutes. Method was developed based upon the fact that the crushed IM tablet particles did not exhibit weight change when heated at 160° C. for more than 30 minutes. So, the selected temperature and time settings made sure that the tablet mass is dried completely at the end of the test program.

### 2.2.2.7 Scanning Electron Microscopy (SEM)

Tablet morphology was visualized by a scanning electron microscope (Jeol-6060, Tokyo, Japan). Tablet samples were fragmented and then gold sputtered for 60 seconds to minimize the charging effects. Micrographs were obtained at an acceleration voltage of 15 kV.

### Results and Discussion

### 3.1 XRD and DSC Analysis of Formulation Constituents and IM Tablets

XRD and DSC analysis shows the crystalline properties of griseofulvin, xylitol and lactose and the amorphous nature of maltodextrin. Distinct diffraction peaks were observed at 2θ of 0.91°, 13.38°, 14.70°, 16.62°, 23.96°, 26.78° and 28.65° in XRD pattern of griseofulvin. Xylitol had sharp multiple diffraction peaks at 2θ of 13.86°, 14.52, 17.59°, 19.81°, 22.15°, 22.46°, 24.62°, 28.05° and 31.53°. Peculiar peaks were observed for lactose at 2θ of 10.49°, 19.09° and 20.95°. A broad halo scattering profile was obtained for maltodextrin, confirming its amorphous form. A perfect concordance between the XRD diffraction patterns of IM tablets and grisoefulvin confirmed that griseofulvin remained crystalline in nature in IM product. Specific griseofulvin diffraction peaks can be observed at 2θ of 10.91°, 13.38°, 14.07°, 16.62°, 23.96°, 26.78° and 28.65° in IM tablets. Xylitol and lactose got converted into an amorphous form in the IM tablets.

DSC thermograms of xylitol, griseofulvin, and lactose showed melting endotherms at 95.69° C., 220.1° C. and 235.7° C. respectively, indicating the crystalline nature of these constituents. Maltodextrin is amorphous in nature without any melting endotherm. DSC thermogram of IM product showed a glass transition temperature at 84.65° C. and a melting endotherm of griseofulvin at 212.94° C., further confirming its crystalline status in a solid dispersion. The absence of melting endotherms for xylitol and lactose provided additional evidence of their conversion to an amorphous state in the IM product. Although griseofulvin is dispersed as crystals, a decrease in the griseofulvin melting point could be expected due to the polymer-drug interaction and/or decrease in drug particle size with HME processing, resulting in a melting point depression of griseofulvin.

### 3.2 Polarized Light Microscopy

Polarized light microscopy is an easy and sensitive method to qualitatively observe crystalline domains within a polymer matrix. These crystalline domains were well dispersed in amorphous polymer regions. This imaging study confirmed dispersed griseofulvin crystalline domains in an amorphous polymer matrix.

### 3.3 In Vitro Release Studies

The maltodextrin matrix provided an immediate release profile of the drug, even though it maintained crystalline property of drug particles. For the formulated griseofulvin tablets, 95% drug release was achieved in 20 minutes. As per the griseofulvin tablets—USP monograph, Dissolution Test 1, at least 75% of the labeled amount of griseofulvin should be dissolved in 90 minutes. The results established that the IM griseofulvin tablets significantly outperformed the dissolution specifications of the monograph. In comparison, the marketed product takes between 50 to 60 minutes for 75% release under similar sink conditions.

### 3.4 Effect of CPPs on IM Tablet Quality

From the preliminary studies, it was observed that the injection pressure and mold surface temperature significantly affect the quality of IM tablets and were studied in more details.

### 3.4.1 Injection Pressure

Sufficient injection pressure is required to inject the molten material in the mold cavity areas. Along with hold pressure, injection pressure is a critical parameter. In the current study, injection pressures of 1300 psi and 1630 psi were found to be insufficient to provide uniform, fully filled mold cavities. In the injection molding field, this phenomenon is sometimes referred to as a short shot. Insufficient injection pressure is one of the plausible reasons for a short shot. Insufficient filling will also leave gaps between cooling parts and molds resulting in uneven cooling of the injection material. Therefore, the molded part will not shrink uniformly, sometimes leaving surface depression; this phenomenon has been coined as a sink mark. Sink marks are areas in a molded part where the surface is deformed into a depression. At these lower injection pressure values, nonuniform and peculiar shaped tablets were obtained and could be called “sink marked” tablets. Weights of these “sink marked” tablets were low, 490±14 mg. Increasing the injection pressure to 1960 and 2285 psi resulted in uniform and fully filled molded tablets. Weights of these fully filled molded tablets were 524±1 mg. Increasing the injection pressure to even higher values (2610 and 2940 psi) resulted in flashing, another common problem of injection molded parts. This flashing results in increased weight variation and friability problems. It would also lead to protrusions outside the normal tablet surface and will affect an optional tablet coating step. Based upon the results, 1960 psi was selected and used for the next series of experiments. [Note: as per single parameter method, all 4 selected mold surface temperatures (30, 35, 40 and 45° C.) were employed in this study (discussed herein) and same phenomena, like short shot, sink marks and flashing, were observed at each temperature setting at lower or higher pressures].

### 3.4.2 Mold Surface Temperature

In IM processing, mold temperature was controlled by a continuous cooling system, where coolant (propylene glycol-water mixture), maintained at the selected temperature, was circulated in the cooling channels to remove the heat. This continuous cooling system cools the mold surface and injected polymer and then generates a congealed polymer layer at the mold cavity surface. As the mold cavity gets continuously filled during injection step, this solidified layer would further stiffen and increase the melt flow resistance and decrease the mold-filling ability. This could also be an additional reason why the material injected at 1300 and 1630 psi could not fill the mold cavities completely and resulted in tablets with sink marks. As mentioned in Table 1, the mold surface temperature was fixed at one particular temperature, and tablets were manufactured to study the effect of mold surface temperature on tablet quality attributes. At a mold temperature of 30° C., the IM process produced broken and chipped tablets more frequently. With an increase in mold surface temperature (35° C. and 40° C.), there was continued improvement in product quality. At 45° C., the injection molding process yielded tablets without cracks and chipping issues. High values of mold surface temperature resulted in a smaller difference between the surface mold temperature (highest used 45° C.) and melt temperature (130° C.). It led to a slower cooling rate. Because of this increase in mold surface temperature, cooling time was gradually increased from 15 s to 30 s. This increase in tablet residence time further increased annealing time. A high mold surface temperature would have significant positive effect on yield stress and reduce the residual stress inside the molded tablets. Because of this lower level of residual stresses, the resultant molded products would have better impact resistance, stress-crack resistance and fatigue performance. Overall, the reduced temperature gradient between the melt temperature and the mold surface and longer residence time rendered an annealing effect and reduced thermal induced residual stress in the tablet, resulting in high quality product without chipping and cracking. As observed in this study, the mold surface temperature had a profound effect on the mechanical properties of the tablets.

### 3.5 Effect of Storage Conditions on Injection Molded Tablets

Tablets stored in open containers at lower humidity (<10% RH) lost 0.5% water content in 2 weeks and maintained that moisture level for 16 weeks. Tablets stored in open containers at 25° C./45% RH and 30° C./65% RH showed increase in % weight, ˜1% and 10% respectively. The tablets remained stable at relatively low humidity and room temperature (19° C.). However, xylitol experienced putative phase separation from maltodextrin and finally phase transition (amorphous to crystalline) when stored at higher temperatures (25° C. and 30° C.) and relatively high humidity (45% RH and 65% RH). Both DSC and XRD showed crystallization of xylitol with time when tablets were stored at 45% RH and 65% RH. The study confirmed that the solid dispersion remains stable at room temperature and relatively low humidity but the plasticizer xylitol experiences phase transition when the product is stored in accelerated stability conditions.

With HME processing, we obtained a miscible dispersion of maltodetrxin and xylitol wherein a homogeneous single amorphous phase, xylitol (originally crystalline in nature) and maltodextrin (polymer) molecules were mixed intimately at a molecular level. Such miscible dispersions can maintain the crystalline molecule in amorphous form due to the reduced molecular mobility and kinetic inhibition of crystallization by the polymer. The amorphous form of the constituent has higher enthalpy, entropy, free energy, and volume as compared with the crystalline form. Since the crystalline form is more thermodynamically stable, the system is metastable, and therefore, there is a thermodynamic driving force for a phase transition. Typically, the glass transition temperature has been used as a molecular mobility gauge and stability has been associated with the difference in glass transition temperature and storage temperature. Formulation storage at high temperature decreases this temperature difference and further leads to higher molecular mobility of the system, that would result in recrystallization. Also, a miscible maltodextrin-xylitol mixture would have a higher enthalpy and free energy and at high temperature, this miscible mixture could de-mix, causing the separation and recrystallization of xylitol from maltodextrin polymer base. Moisture is another factor, influencing phase separation and possible transition of a solid state as well as lowers the glass transition temperature. The extent of moisture uptake mainly depends upon hygroscopicity of the constituents and the storage temperature. The absorbed moisture could work as plasticizer, decrease the viscosity of amorphous phase and increase the molecular mobility of the system. In this study, temperature and moisture both worked synergistically, resulting in the phase transition of xylitol. The main purpose of this study was to monitor the solid-state stability of formulation and direct effect of temperature and moisture on phase separation and phase transition phenomena. Suitability of the formulation as a dosage form was further studied (discussed in section 3.9) by storing the IM tablets in HDPE pharmaceutical bottles at identified storage conditions.

### 3.6 Effect of Tablet Residual Moisture on its Dimensional Stability

In initial studies, all tablet batches were prepared with formulation constituents as received. Long-term tablet storage study confirmed that the tablets showed deviations in dimensions, and there was a significant increase in % tablet thickness with time. These tablets were stored in low humidity conditions at 19° C., and % weight change values confirmed that the tablets did not absorb moisture. Therefore, temperature and humidity were not responsible for these dimensional deviations. Also, constituents did not show any phase transition (amorphous to crystalline form) at this storage condition. After carefully studying the properties of formulation constituents, it was realized that maltodextrin had a high initial moisture content (4.93±0.11%). The moisture content of IM tablets, immediately after manufacturing, was measured to be 1.96±0.17%. Given this, maltodextrin was first dried in an oven at 85° C. until it attained significantly low moisture content (0.6±0.2%) and then used for tablet manufacturing. The resultant manufactured tablets had low residual moisture (0.59±0.14%). These tablets were then placed in long-term stability testing at the same storage conditions (19° C., <10% RH), and tablet dimensions were measured. Tablets with low residual moisture did not show any dimensional deviations. The study confirmed that the residual moisture played a critical role in IM tablet expansion. This study confirmed that the residual moisture present inside the tablet probably induced phase separation of maltodextrin and xylitol and resulted in tablet expansion. These experiments were replicated thrice for further confirmation, and resultant IM tablets were stored again for long-term stability at 19° C. and <10% RH, giving the same results each time. To confirm maltodextrin-xylitol phase separation, mDSC studies were carried out.

### 3.7 Phase Separation Study by Modulated DSC

### 3.7.1 Effect of Long-Term Storage on Glass Transition (and Thus Phase Separation) of Tablets

A broadening of glass transition temperature (DSC peak width) corresponds to multiple phases. When tablets with high residual moisture content were stored for 20 weeks at 19° C., <10% RH, a broadening of the glass transition event indicated a significant increase in maltodextrin-xylitol phase separation in comparison with tablets having low residual moisture content. Also, enthalpy relaxation (in glass transition temperature range) was calculated from the nonreversible heat flow signal for all tablet samples (Table 14). A significant increase in the enthalpy relaxation value (for tablets with high residual moisture, long-term storage) further indicated significant phase separation.

TABLE 14
Enthalpy relaxation and AUC values of derivative of reversible heat flow
patterns obtained from DSC study

| | Enthalpy | AUC, Derivative |
|---|---|---|
| | relaxation | of reversible |
| | (Tg range, | heat flow |
| Tablet type with storage time | J/g) | (W min/g ° C.) |
| Tablets, low residual moisture, 0 week | 2.13 | 0.00217 |
| Tablets, low residual moisture, 20 weeks | 1.96 | 0.00466 |
| Tablets, high residual moisture, 0 week | 2.07 | 0.00384 |
| Tablets, high residual moisture, 20 weeks | 36.51 | 0.0117 |

### 3.7.2 Effect of Long-Term Storage on the Derivative of Reversible Heat Flow Patterns of Tablet Samples

The temperature derivative of the reversible heat flow signal was calculated. For this derivative, a step change in heat capacity would appear as a peak. A higher number of peaks and larger deviations from zero indicates phase separation. The tablets with high residual moisture showed peaks and significant fluctuations in signal pattern compared to the tablets having low residual moisture content (stored for same time period). Moreover, there was an increase in the area under the curve (AUC) of the derivative reversible heat flow signal (for stored tablets having high residual moisture content) due to the changes in signal patterns. These observations pointed towards the phase separation of maltodextrin and xylitol when residual tablet moisture is high.

In the absence of moisture, the molecular mobility was very low for dried IM tablets at 19° C. and low humidity storage conditions. Xylitol, therefore, continued to exist in a kinetically frozen state of miscibility with maltodextrin. For tablets having high residual moisture, phase behavior in the solid state would have become complex in nature. It can be concluded that the presence of moisture resulted in high molecular mobility and self-association of the xylitol and maltodextrin to each other, and ultimately xylitol rich and maltodextrin rich phases were separated.

### 3.8 Scanning Electron Microscopy (SEM) of Shattered Tablet Parts

In the case of tablet made with maltodextrin, an excess of trapped water vapor in the polymer matrix might exist as microbubbles in the molten material at the required water content (e.g., more moisture than the formulation could solubilize), temperature and pressure. As the molten material cooled and solidified in the molds, the microbubbles could become trapped and solidify if conditions remained favorable to their continued existence as bubbles. A SEM study confirmed the presence of microbubbles in the high moisture tablet polymer matrix. On the other hand, tablets made with pre-dried maltodextrin showed no such microbubbles. Thus, SEM study further clarified the importance of eliminating excess moisture in HME-IM.

### 3.9 Long-Term Formulation Storage Study in Closed Containers

The plasticizer xylitol showed a phase separation and phase transition when stored in accelerated storage conditions in open containers. Thus, it became imperative to confirm the stability of IM tablets when stored in pharmaceutical packaging (sealed bottles) in accelerated storage conditions. IM tablets were packed and stored in 6 different storage conditions (19° C./<10% RH; 19° C./23% RH; 19° C./53% RH; 25° C./45% RH; 30° C./65% RH and 40° C./75% RH) for 15 weeks. IM tablets, when packed in sealed bottles, did not uptake a significant amount of water except when stored at 30° C./65% RH and 40° C./75% RH. The formulation was dimensionally stable in all storage conditions. Considering solid state properties, xylitol started crystallizing out only when stored at 40° C., 75% RH. Typical pharmaceutical packaging improved the physical stability, and the formulation should be robust for pharmaceutical commercialization.

### Conclusion

Injection pressure, mold surface temperature, storage conditions and residual tablet moisture showed a significant impact on the physical stability of IM tablets. Lower injection pressure (<1630 psi) resulted in insufficient mold cavity filling and showed sink marks on tablet surface, whereas, higher injection pressure (>2610 psi) resulted in flashing. Operating within an optimized injection pressure range (1960 to 2285 psi) yielded robust tablets. Higher mold surface temperature increased tablet cooling time (from 15 s to 30 s) in mold cavities and reduced temperature gradient (from 90° C. to 75° C.) between melt temperature and mold surface temperature. This reduced temperature gradient and increased residence time further minimized residual thermal stress, rendered annealing effect and prevented tablet chipping and cracking.

Dimension measurement of IM tablets revealed that the tablets possessing high residual moisture expanded significantly with time, whereas, tablets possessing low residual moisture did not change their dimensions when stored in low humidity conditions (<10% RH) at ambient temperature. The residual moisture in IM tablets from formulation constituents played a critical role in IM tablet expansion. DSC analyses proved that the tablets with high residual moisture had maltodextrin-xylitol phase separation, whereas this was not the case for tablets with low residual moisture. Thus, phase separation can be linked to dimensional deviations and should be avoided in IM tablets by eliminating possible entry of moisture in solid dispersion. SEM further underpinned the HME-IM processing of formulation constituents possessing variable moisture contents and resultant IM tablet microstructure. The formulation was found to be stable when stored in typical pharmaceutical packaging. This study further proved that an integrated HME-IM technology platform is a promising platform to manufacture pharmaceutical tablets in a continuous mode and provides robust tablet formulation when identified CPPs and formulation attributes affecting tablet quality attributes are taken care of.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element or a list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for coating a pharmaceutical tablet, comprising;
positioning a tablet core in a first orientation in an injection mold cavity;
injecting a coating composition into the injection mold cavity to form an injection-molded coating on a first portion of the tablet core;
reorienting the tablet core with respect to the injection mold cavity; and
injecting the coating composition into the injection mold cavity to form an injection-molded coating on a second portion of the tablet core to produce a coated pharmaceutical tablet, wherein the injection-molded coating is substantially continuous.

2. The method of claim 1, further comprising forming the tablet core through injection molding prior to the step of positioning the tablet core in the injection mold cavity.

3-5. (canceled)

6. The method of claim 1, further comprising extruding the coating composition prior to injecting the coating composition into the injection mold cavity.

7. The method of claim 1, wherein the second portion of the injection-molded coating overlaps the first portion of the injection-molded coating.

8. A method for manufacturing a coated pharmaceutical tablet, comprising;
injecting a coating composition into an injection mold cavity to form a first portion of an injection-molded coating;
injecting a tablet core composition into the injection mold cavity onto the first portion of the injection-molded coating to form a partially-coated tablet core; and
injecting the coating composition into the injection mold cavity onto the partially-coated tablet core to form a second portion of the injection molded coating and to produce a coated pharmaceutical tablet, wherein the injection-molded coating is substantially continuous.

9. The method of claim 8, further comprising solidifying the first portion of the injection-molded coating, prior to injecting the tablet core composition into the injection mold cavity onto the first portion of the injection-molded coating.

10. The method of any of claim 9, further comprising solidifying the partially-coated tablet core, prior to injecting the coating composition into the injection mold cavity onto the partially-coated tablet core.

11. (canceled)

12. A coated pharmaceutical tablet, comprising:
a tablet core; and
an injection-molded coating surrounding the tablet core, wherein the injection-molded coating is solvent-free and substantially continuous.

13. The coated pharmaceutical tablet of claim 12, wherein the tablet core comprises an injection-molded tablet core.

14. The coated pharmaceutical tablet of claim 12, wherein the coating composition comprises at least one polymer and at least one plasticizer.

15. The coated pharmaceutical tablet of claim 14, wherein the coating composition comprises 50% to 100% by weight polymer, and 0% to 50% by weight plasticizer.

16. The coated pharmaceutical tablet of claim 14, wherein the polymer comprises polyethylene oxide and the plasticizer comprises polyethylene glycol.

17. The coated pharmaceutical tablet of claim 14, wherein the polymer comprises an acrylate-based polymer and the plasticizer comprises an acrylate-based plasticizer.

18. The coated pharmaceutical tablet of claim 12, wherein the coating composition comprises a polyvinylcaprolactam-based graft copolymer.

19. The coated pharmaceutical tablet of claim 12, wherein the coating composition comprises a polyvinyl alcohol/polyethylene glycol graft copolymers.

20. The coated pharmaceutical tablet of claim 14, wherein the polymer comprises polyvinyl alcohol and the plasticizer comprises glycerine or polyethylene glycol plasticizer.

21. The coated pharmaceutical tablet of claim 14, wherein the polymer comprises a graft copolymer of polyvinyl alcohol and polyethylene glycol and the plasticizer comprises glycerine or polyethylene glycol.

22. The coated pharmaceutical tablet of claim 14, wherein the polymer comprises polyethylene glycol and a graft copolymer of polyvinyl acetate and polyvinylcaprolactame-based polymer and the plasticizer comprises at least one of glycerine or polyethylene glycol.

23. The coated pharmaceutical tablet of claim 12, wherein the injection-molded coating has an average thickness of 150 to 300 microns.

24. The coated pharmaceutical tablet of claim 12, wherein the coating composition has a Young's modulus of less than 700 MPa, an elongation of greater than 30%, a toughness of greater than 95×10⁴ J/m³, and a melt flow of greater than 0.4 g/min.
